SEQUENCE LISTING (44 sequences)

SEQ ID NO: 1 - DNA, 795 bp, Pyrococcus furiosus
atgaagaaaggagcaattggtatcggaacgctcatcgtcttcatcgcaatggtgcttgttgcggcagtagcagcaggtgtgctaatagcaacaagtggatatttgcagcagaaggccatggccacaggtagacagacaacccaggaggttgcaagtggaatcaaggttactggtgtgttcggctatatcaatggcactccccctggagcctcaaacataagcaggattgtcatatatgttgctccaaatgcagggagtagtggaattgacttaagatatgtaaaaatagtgttaagcgatgggaaaagaatggcagtgtacaggtattacgatccaaaggaggatggaagctcagacctaaagccagaatacattcactacaaaggagatatacctaacatatttgcttatggagagtgggaaccctactacaaaaacaagaagccacagatatctggagaatacatcaccgataatattaacgtaagtgcagtttggtggaacctctacagtgcctacaacaactcaagcaagctactcttcgggattgcggtagttcaagatggggacaacagccttagcgatccacaacatccaacattaagctggggagacttagcagccctaatgatatggactttcccattcgacgatgacaataatatctccaacggtttcgggctaagaccaggaacaaagattataggaaaggtaattccagagagcggagctgctggtgttatcgacttcacaactccctctacatatacccaaaacttaatggaacttcaatga

SEQ ID NO: 2 - protein, 263 aa, Pyrococcus furiosus
MKKGAIGIGTLIVFIAMVLVAAVAAGVLIATSGYLQQKAMATGRQTTQEVASGIKVTGVFGYINGTPPGASNISRIVIYVAPNAGSSGIDLRYVKIVLSDGKRMAVYRYYDPKEDGSSDLKPEYIHYKGDIPNIFAYGEWEPYYKNKKPQISGEYITDNINVSAVWWNLYSAYNNSSKLLFGIAVVQDGDNSLSDPQHPTLSWGDLAALMIWTFPFDDDNNISNGFGLRPGTKIIGKVIPESGAAGVIDFTTPSTYTQNLMEL

SEQ ID NO: 3 - DNA, 582 bp, Halobacterium sp.
atgttcgagttcatcactgacgaagacgagcgcggccaagtggggatcggcacgctcatcgtgttcatcgcgatggtgctggtcgccgcgatcgccgccggcgtcctcatcaacaccgccggctacctccaatccaaggggtcggcaaccggtgaggaagcctccgcacaggtctccaaccgcatcaacatcgtctccgcgtacggcaacgtcaacaacgagaaggtcgactacgtgaacctcaccgtgcgccaggccgccggagccgacaacatcaacctcacgaaatccacgatccagtggatcggcccggacagagccaccaccctgacgtactcgtcgaacagcccgagttcgctgggtgaaaacttcaccaccgaatccatcaagggcagcagcgccgacgtgctggtcgaccagtccgaccgcatcaaggtcatcatgtacgccagcggcgtcagctccaacctcggcgctggtgacgaggtgcagctgacggtgaccacgcagtacggctcgaaaaccacctactgggcgcaagtccctgaatcgctcaaggacaaaaacgccgtcacactataa

SEQ ID NO: 4 - protein, 193 aa, Halobacterium sp.
MFEFITDEDERGQVGIGTLIVFIAMVLVAAIAAGVLINTAGYLQSKGSATGEEASAQVSNRINIVSAYGNVNNEKVDYVNLTVRQAAGADNINLTKSTIQWIGPDRATTLTYSSNSPSSLGENFTTESIKGSSADVLVDQSDRIKVIMYASGVSSNLGAGDEVQLTVTTQYGSKTTYWAQVPESLKDKNAVTL

SEQ ID NO: 5 - DNA, 519 bp, Halobacterium sp.
atggtgctggtcgccgcgatcgccgccggcgtcctcatcaacactgccggctacctccaatccaaggggtccgcaactggtgaggaagcctccgcacaggtctccaaccgcatcaacatcgtctccgcgtacggcaacgtggacacgtctggctcaaccgaggtagtcaattacgcgaacctgacggtgcgccaggccgctggggctgacaacatcaacctcagcaaatccacgatccagtggatcggcccggacaccgccactaccttgacctacgacgggactactgccgacgccgagaacttcaccacgaattcgattaagggcgacaacgcggacgtgctggttgatcagtccgaccgcatcgagatcgtcatggacgcggccgagatcaccaccaatggactgaaggctggcgaagaggtccagctgacagtgaccacgcagtacggctcgaaaaccacctactgggcgaacgttcctgagtcgctcaaggacaaaaacgcagtcacgctataa

SEQ ID NO: 6 - protein, 172 aa, Halobacterium sp.
MVLVAAIAAGVLINTAGYLQSKGSATGEEASAQVSNRINIVSAYGNVDTSGSTEVVNYANLTVRQAAGADNINLSKSTIQWIGPDTATTLTYDGTTADAENFTTNSIKGDNADVLVDQSDRIEIVMDAAEITTNGLKAGEEVQLTVTTQYGSKTTYWANVPESLKDKNAVTL

SEQ ID NO: 7 - DNA, 648 bp, Methanococcus vannielii
atgagtgtaaaaaatttcatgaataacaagaaaggtgactctggaatcggcaccttgattgttttcattgcaatggtattggttgctgcagttgcagcaagtgttttaattaacacaagtggatttttacagcaaaaagctgcaacaacaggaaaagaaagtactgaacaggttgcaagtggattacaagtaatgggcgtaaatggataccaggatggaactaatgatgcaaatgtaagtaaaatggcaatttatgtaacccctaacgcaggaagttcagcaattgaccttacaaattcaaaattatttgtaacctacgatggccagacccacgtcttagcttacgatgacgttacagaccttacaacaggtaattcagatattttcgatgcaattaatgttggaacccctgcttctgaattccacgttgcagtactccaggataatgataattcaactggaaatggagtaattaataaaggagatattgtagcaatagtaattgaaactagcgacatttttggcaatgacggaattcctgaaagaaagagtgtttctggaaaagtacaaccggaatttggtgctccaggagtatttgaattcacgacacctgcaacgtacactaacaaggtattggaattacaataa

SEQ ID NO: 8 - protein, 215 aa, Methanococcus vannielii
MSVKNFMNNKKGDSGIGTLIVFIAMVLVAAVAASVLINTSGFLQQKAATTGKESTEQVASGLQVMGVNGYQDGTNDANVSKMAIYVTPNAGSSAIDLTNSKLFVTYDGQTHVLAYDDVTDLTTGNSDIFDAINVGTPASEFHVAVLQDNDNSTGNGVINKGDIVAIVIETSDIFGNDGIPERKSVSGKVQPEFGAPGVFEFTTPATYTNKVLELQ

SEQ ID NO: 9 - DNA, 606 bp, Natrialba magadii
atgttcgaacaaaacgacgaccgcgaccgtggtcaggtggggattggcacccttatcgtgttcatcgcgatggtgcttgtcgctgcgattgccgcgggcgtgctgatcaatacggctggcatgctgcagacgcaggcagaagccaccggtgaagagagtacagatcaagtaagtgaccgcctggacatcgtcagtgtctcaggggatgttgatgatcccgatgaccctactcaaatcaacaacatcagtatggtgactgcgactgcgccgggatcggatccagttgacttgaatcaaacaacggcgcagttcatcggtgagggtggtgaagagatgtttaatcttagccacgagggcgtcttcatcaacagcatccaaggcgtcacggatgaacccgataacaacgtcttgacggaaagttcggaccgtgctgaagttgtgttcgaattagacggagccccaggtagttacgatattggctacgaagcattggatgagagtgaacggttgacggttatcctgacgactgacgccggtgcgtccaccgaacaggagattcgcgttccaagtaccttcattgaagacgaagaatcggtgagactgtag

SEQ ID NO: 10 - protein, 201 aa, Natrialba magadii
MFEQNDDRDRGQVGIGTLIVFIAMVLVAAIAAGVLINTAGMLQTQAEATGEESTDQVSDRLDIVSVSGDVDDPDDPTQINNISMVTATAPGSDPVDLNQTTAQFIGEGGEEMFNLSHEGVFINSIQGVTDEPDNNVLTESSDRAEVVFELDGAPGSYDIGYEALDESERLTVILTTDAGASTEQEIRVPSTFIEDEESVRL

SEQ ID NO: 11 - DNA, 780 bp, Natrialba magadii
atgttcactaacgacaccgacgacggccgcggtcaggtggggatcggcacgctcatcgtgttcatcgcgatggtgctggtcgctgcgattgctgcgggcgtcctgatgaacacagctgggatgttgcagtcccaggctgaagcaactggtgaagagagtaccgaccttgtctctgaacggatcgataccacgatcgcagtgggtaccgtatccacccatgtggcagacggtgaagacggtgcagatcgcggtgacttagcggagatcagtattggcgttaccggtgcacccggggcagatgatattgacctcaatgagacgataattcaggtcgtcggtcctgagggggcagagaatctcgtcatggctgacggaagcaatgacatgagtgaagctgggtgggacgaaactagcaccaccgacattgggagtactgagagtactgaccaaggagatactgacgacgacgtaaacgcctcaaacatcgagagcggatacttcgctgtcgaaaacgaagacggatactttgtcgagggtagcgatgcagtcctcgatgacaacaatggcgaactcacgatcgtcttcaatccaaaagtcgcaccatttggtgaggctgatgatgtaagcggcatcacccctggagatcttcatgaagatgacgtcttcggtgcgggcgacgaggcctcggtcgacatcgtctcgccatccggtgcaaccacctcggtcgaactgaactccccagacctcttcagcgagcctggtgaagcggtccgactctaa

SEQ ID NO: 12 - protein, 259 aa, Natrialba magadii
MFTNDTDDGRGQVGIGTLIVFIAMVLVAAIAAGVLMNTAGMLQSQAEATGEESTDLVSERIDTTIAVGTVSTHVADGEDGADRGDLAEISIGVTGAPGADDIDLNETIIQVVGPEGAENLVMADGSNDMSEAGWDETSTTDIGSTESTDQGDTDDDVNASNIESGYFAVENEDGYFVEGSDAVLDDNNGELTIVFNPKVAPFGEADDVSGITPGDLHEDDVFGAGDEASVDIVSPSGATTSVELNSPDLFSEPGEAVRL

SEQ ID NO: 13 - DNA, 1187 bp, Natrialba magadii
atgttcacatccaatacagatgacgaccgtggccaggtggggatcggtacgctcatcgtgttcatcgcgatggtgctggtcgctgcgattgctgcgggcgtattgatcaatacggctggcatgctgcagacgcaggccgaagccaccggcgaagagagtacagatcaggtaagtgaccgacttgaaatctcgagtacgtctggagatttcagtgacgtaaatacccttggtgccggtgaaggcgaagaattggaggtaacggttgaagccggtgacgctacggcagcaggcgaagaagtcgtaataagagttgcaacaagtgctgaatctggatttgaggactcgaaggcaatcgaattacctgatgaagccggagatccaacaactgtcacacttgataatcttccttcaataggtggtgcattggtaactgttgatggagaaaatgttcaagcagtaacggaagacagtgtggacctcactcaaggagatccaagcgtcagttttaacgtagacgaactcaggatgattccgagtcgactattggtcttcaactcaccgctgatgctggtaacaacttctgggacgaaatagcggaagataatatcgaagataccgttactgttcagttgaccgactacgagcgtactgaagctgaaataacgaacgtgaataattggggcagtgatgacgcagagattgagtgggaagcaacggtgccggctgacgagggagactatgcagtggaagtaataggattcgactcagcacggatgcttccaatttcgacaaatgaggtagcaagtacaacggaagatccagaacttggtgaaactgacactcaaatcgacaaccttcagttctctgtcgctactgcacctggctctgacgcgatcgatcttgaggagacgtcagtgcagttcatcggtgatcagggcgaggagacggttacgatcactgaccggaacgtcgagaacatccagggtgtcgacggaaacgtcctgacggataattccgatcgtgcactcgtctcgttcgacccagtcgccgacattgacggattcaaccgaatcgaagagagcgaggacctcaccgtcatattcacgacggcatcgggagcctcgacagagaccgaactacgcattccaagcaccttcctcgaaggtgacgaatctgtgaggctataa

SEQ ID NO: 14 - protein, 395 aa, Natrialba magadii
MFTSNTDDDRGQVGIGTLIVFIAMVLVAAIAAGVLINTAGMLQTQAEATGEESTDQVSDRLEISSTSGDFSDVNTLGAGEGEELEVTVEAGDATAAGEEVVIRVATSAESGFEDSKAIELPDEAGDPTTVTLDNLPSIGGALVTVDGENVQAVTEDSVDLTQGDPSVSFNVDELSDDSESTIGLQLTADAGNNFWDEIAEDNIEDTVTVQLTDYERTEAEITNVNNWGSDDAEIEWEATVPADEGDYAVEVIGFDSARMLPISTNEVASTTEDPELGETDTQIDNLQFSVATAPGSDAIDLEETSVQFIGDQGEETVTITDRNVENIQGVDGNVLTDNSDRALVSFDPVADIDGFNRIEESEDLTVIFTTASGASTETELRIPSTFLEGDESVRL

SEQ ID NO: 15 - DNA, 786 bp, Natrialba magadii
atgtttgtcaacgaaactaccgacgaccgcggccaagtggggatcggtacgctcatcgtgttcatcgcgatggtgctggtcgctgcgattgccgcaggtgtactgatcaacacggccgggatgctgcaatcccaggccgaagcaaccggtgaggagagtaccgatctcgtttccgaacggatcgattcaacgactgcagtcggtattgtctccgaaaccgaagttagcgaggaggctggtgccgaccgaggtgaactcgaagagattcgtcttggcgtcagcggtgctgctggctccgacaatattgacctcagtgaaaccatcattcaggttgtgggccctcaaggacaggataaccttgtgatggctgatcctggtgatgatgaaatcgatgccaatgatgatggattcgtcacggtgactgacgaagatggtaatgtggatggagacagtactgatgcaactgatgctgacccatcccacattgcgtctggacacttcgccgttgaaaacgaagatggcaatttcgtcgaggaaagcgatgcagtcctcgataacgacaacggcgaactcacgattatcctcaatccgaaggtagcaccgttcggatcgcaaataagcgaaagtgatgaggaactagatctgcaggatctggacactgaggacgccttcggtgctggagacgaatcctctctcagtatcgtttcgccatccggtgcaacgacggaggtcgaactgaacgcgcctgacctcttcagcgaggacggcgaagcagttcgcctctaa

SEQ ID NO: 16 - protein, 261 aa, Natrialba magadii
MFVNETTDDRGQVGIGTLIVFIAMVLVAAIAAGVLINTAGMLQSQAEATGEESTDLVSERIDSTTAVGIVSETEVSEEAGADRGELEEIRLGVSGAAGSDNIDLSETIIQVVGPQGQDNLVMADPGDDEIDANDDGFVTVTDEDGNVDGDSTDATDADPSHIASGHFAVENEDGNFVEESDAVLDNDNGELTIILNPKVAPFGSQISESDEELDLQDLDTEDAFGAGDESSLSIVSPSGATTEVELNAPDLFSEDGEAVRL

SEQ ID NO: 17 - DNA, 668 bp, Pyrococcus abysii
atgaggagaggtgcgatcggcattggcacgttgatagttttcatcgcaatggttttagtagcggcagtagcagcgggagtgctcattagcacttctggatatctccagcaaagggcaatgtctgtaggcctagagactacaagggatgtttcaagtggtctcagaataatctcaatctggggctatgcccctaagaatactactggcaataccaccattcagagcaatattaccaaactcgccatatacatagctcccaacgctggaagtgaacccataaacctcaaccagacaaggataatactcacagtaaagtcaacgatggtcatatttacctttggtggagaggataccgttgcagactggacgaatggtgcagttaatgtctttaatgaaaccatatgggaaaatattaacggaacaaagtttggagtgggagttgtggttgatagcgataaaagcatgctttccaacaaggcatcaccgggaatgaactcgggagatttagcagtactgctaattaacactaaattggcttttaacaaatacgggggaattccgcctaacacaaaggtggtcggtaagatactgccaccacacggtgcaggaactgttatcgacttaataactccagctacttactccagtgagggtattgagctccagtg

SEQ ID NO: 18 - protein, 222 aa, Pyrococcus abysii
MRRGAIGIGTLIVFIAMVLVAAVAAGVLISTSGYLQQRAMSVGLETTRDVSSGLRIISIWGYAPKNTTGNTTIQSNITKLAIYIAPNAGSEPINLNQTRIILTVKSTMVIFTFGGEDTVADWTNGAVNVFNETIWENINGTKFGVGVVVDSDKSMLSNKASPGMNSGDLAVLLINTKLAFNKYGGIPPNTKVVGKILPPHGAGTVIDLITPATYSSEGIELQ

SEQ ID NO: 19 - DNA, 660 bp, Pyrococcus abysii
atgcacagaaagggtgcaataggcataggaacgctcattgtcttcattgcaatggttctagtagcggcagtagcggcgggagttatcattggaacagctggttatcttcaacagaaggcacaggctacaggcatgcagacaacccaagaggtttccagtgggataaagatcatcaacatctatggttacgtaaactcctctgtccctagtaatggcacaataaccaagatggcaatattcgtctcacctaacgcagggagtggggggatatccctcagtaacgtgaaaattgttctcagcgatggcaagaaactcgttgtctataattatagcaagggattgctttatgacaaacagataagcgacttgttcaatgattctatcgttacgatatggaacaacattaccgatacaaccttcggaatagcggtcattaacgacagtgggaacaaaatggacaaagattatccaaacttagaatggggagataccgtggcactactcctcaggacaacagtttttgaaacagaggataaccgtagaggaatcggtcctggtactaggatagttgggaaggtaattcccgaagttggggctgcaggtgttatagacttcacaacaccctcaacatataactaccgggtgatggtactccagtga

SEQ ID NO: 20 - protein, 219 aa, Pyrococcus abysii
MHRKGAIGIGTLIVFIAMVLVAAVAAGVIIGTAGYLQQKAQATGMQTTQEVSSGIKIINIYGYVNSSVPSNGTITKMAIFVSPNAGSGGISLSNVKIVLSDGKKLVVYNYSKGLLYDKQISDLFNDSIVTIWNNITDTTFGIAVINDSGNKMDKDYPNLEWGDTVALLLRTTVFETEDNRRGIGPGTRIVGKVIPEVGAAGVIDFTTPSTYNYRVMVLQ

SEQ ID NO: 21 - DNA, 528 bp, Pyrococcus abysii
ttgaaaaacctccaagggggtgcatggcaaatggcaagaagaggtgcgattggtattggtaccctaatagtgtttattgccatggtgttagtggctgcagtagctgcagcagttctcataaacacgagcggcttcctccagactagggcttcaacagtaggtaaggagcagaccaggcaagtttcgactggttttattctcaaggacgcctatgtaacaggcaccaatacgataaaccttctagtaaccctaccaacggggagctatcccgtcgacattagcaggacagttataatcgtaaacggaaagcaactcacatatggtagtactgctaacaccacaaatttctctgcaaaacctctggtaggagagattaacggcgacattgtacaaccaggatcaacaattctcataacattcaatatgagtgagggttggaccgtcgctcggggagaaatcgttcctaacgttggttcaccaactccattcactgtaaccaaagatcttgatagtgttcccagtagctga

SEQ ID NO: 22 - protein, 175 aa, Pyrococcus abysii
MKNLQGGAWQMARRGAIGIGTLIVFIAMVLVAAVAAAVLINTSGFLQTRASTVGKEQTRQVSTGFILKDAYVTGTNTINLLVTLPTGSYPVDISRTVIIVNGKQLTYGSTANTTNFSAKPLVGEINGDIVQPGSTILITFNMSEGWTVARGEIVPNVGSPTPFTVTKDLDSVPSS

SEQ ID NO: 23 - DNA, 708 bp, Pyrococcus horikoshii
atgaggaggggtgctattggtattggaacgctcatcgtgttcatcgcaatggtattggtagctgcggtagctgctggagtgttaattacaaccagtggctaccttcagcagaaggccatggccactggtaggcagaccacccaggaagtagcaagcggaatcagagtgagtggcatttatggctatactccttcaaaccctccaggaagtggaaagataacgaggctagtagtctacgttactccaaacgctggtagcggaggtattgatctcgcccatgttagagttgtattaagtgacggtaaaagaatggcagtgtataggtactatgattcagacaaagaccaaggactccaagcaggctatttcctatatgcaggggatattgagaacatagtaccctactttaacgatacagatgtactctcagtaagcaattatacaacggtaaccagtgtcgctgatgtctggaagaatctatattatgcaatgacacaagacaataagatgctctttggaattgtggtcgttgcagatgacgatgatagcctaagcaatacagctcatcccacgcttgggtttggagacaaagccgccctaatcttgtggacgataccattcgatgacgacaatgattacagcaatggctatggaataccattcacgactccatcgacttacacggataacctaatggagctccagtga

SEQ ID NO: 24 - protein, 255 aa, Pyrococcus horikoshii
MRRGAIGIGTLIVFIAMVLVAAVAAGVLITTSGYLQQKAMATGRQTTQEVASGIRVSGIYGYTPSNPPGSGKITRLVVYVTPNAGSGGIDLAHVRVVLSDGKRMAVYRYYDSDKDQGLQAGYFLYAGDIENIVPYFNDTDVLSVSNYTTVTSVADVWKNLYYAMTQDNKMLFGIVVVADDDDSLSNTAHPTLGFGDKAALILWTIPFDDDNDYSNGYGIPPSTKVVGKVIPENGAGGVIDFTTPSTYTDNLMELQ

SEQ ID NO: 25 - DNA, 945 bp, Pyrococcus horikoshii
gtgaagaaaggtgctgtgggtattggtacccttatagtgtttattgctatggtgttagtggctgcagtagctgctgcagtgctcatcaacacgagcggttacctccagcaaaagagccaggccactggtaggcagaccacccaggaagtagcaagcggaatcaaagtaacaagagttgttggtaaagccgacagtgccaccaatccaacttatattcaagagttagctgtttacataacaccaaatgctggaagctccggaattgacttaactaaggtaaggataactctaagtgatggagccgagctaatgcagaaccttggagctacgataaagttcgataatggaagtgttcaggtgtactttgatccaactgactggacatcagcagcaccaacagtaataattgatacaactaacaaggtcatagagatagtaaatgctactgtagatagtaatgataatcatattaaacctgcgacagacagtaatgtcactataagctttgacactccagtgagcttatatgcctttgctaatccagtcagtgacgtgttcgataatgatgcctttaacaacttaacgactaagactgactttggaatagcagtgcttcaagacagcgatgggagcttagacaacaaggagtatccaaccttaaccaaaggcgatctagtagtactcgctctgagggtaggagggactcagtcattaggatacagctctggagttagcaagatatcagtgatatccacaacaactactgacgttttaacaaagcaatctagcgttaatgtcacaattacatggacagcagtgtttggaaatggattcgacaccggaactaaggttactggaaaagtcattccagaatttggtgctcctggaatcatagagttcacgactccatcaacttacacccagcaggtcattgagcttcagtga

SEQ ID NO: 26 - protein, 334 aa, Pyrococcus horikoshii
MKKGAVGIGTLIVFIAMVLVAAVAAAVLINTSGYLQQKSQATGRQTTQEVASGIKVTRVVGKADSATNPTYIQELAVYITPNAGSSGIDLTKVRITLSDGQKQAIFKYRVGNSANELYFLAELMQNLGATIKFDNGSVQVYFDPTDWTSAAPTVIIDTTNKVIEIVNATVDSNDNHIKPATDSNVTISFDTPVSLYAFANPVSDVFDNDAFNNLTTKTDFGIAVLQDSDGSLDNKEYPTLTKGDLVVLALRVGGTQSLGYSSGVSKISVISTTTTDVLTKQSSVNVTITWTAVFGNGFDTGTKVTGKVIPEFGAPGIIEFTTPSTYTQQVIELQ

SEQ ID NO: 27 - DNA, 624 bp, Pyrococcus horikoshii
atgaggaggggtgctgtgggtattggtacccttatagtgtttattgctatggtgttagtggctgcagtagctgctgcagtgctcatcaacacgagcggttacctccagcaaaagagccaggccactggtaggcagaccacccaggaagtagcaagcggaatcaaagtaacaagtgttattggtcacgtagatacaacgaataatgccatagacaagctagcaatttatgtctcacccaatgctggaagtgaaggtattgacctgagatatactaaaatagttctaaggagcaagagtcaagaggtttcactttactacaaccgcagtaattactacaatggggcagtagataacatatttgacatttcaggagtttggccttcaaatggctacaccttcggaatagttgtcattcaagatagtgacaactcagtccagcagaattatccaacgcttaaccagggagatctggtagcactgactgtaaatgctaatgcagctctcggtggtataaagccaggaacttcaattagtggtgaggttattcctgagcagggtgctcctggcgttatagaattcacaacaccaagcacatacaccgaaactgttgtcgagttacaatga

SEQ ID NO: 28 - protein, 207 aa, Pyrococcus horikoshii
MRRGAVGIGTLIVFIAMVLVAAVAAAVLINTSGYLQQKSQATGRQTTQEVASGIKVTSVIGHVDTTNNAIDKLAIYVSPNAGSEGIDLRYTKIVLRSKSQEVSLYYNRSNYYNGAVDNIFDISGVWPSNGYTFGIVVIQDSDNSVQQNYPTLNQGDLVALTVNANAALGGIKPGTSISGEVIPEQGAPGVIEFTTPSTYTETVVELQ

SEQ ID NO: 29 - DNA, 714 bp, Pyrococcus horikoshii
gtgacagtagtgccaaggaagggtgctgtgggtattggtacccttatagtgtttattgctatggtgttagtggctgcagtagctgctgcagtgctcatcaacactagtggatacttgcaacagaaggcatcggggactggtagagagacaactcaagaagtagcaagcggaatcaaggttgacagagtagtcggttatgctccggacataactggggacataacaagacttgctgtttacatctcaccgaatgccggaagctcagggattgacctaaacaaggttagggtaattctaagcaatggacaaaaggaggtttcccttaagtacaactacgtctataatgctacatccagcacccagacatacgttgcacttccacagggcaacatattcaatgatattgttcttggagtaaatggaaccagtgaaaatgcagcttccacccaggtaaacttcaactggtctctcctgacaggatcaacgttcggtttaatagtgctccaagatgctgacggaagcgtgaaagcaagtactccaactctcaaccagggagaccttgttatcatagctatcgatgtagacgcagcccttggaggaataccaccaaggacttcaattactggtgaggttattcctgagcagggtgctcctggcgttatagaattcacaacaccaagcacatacacggcacatgttatggagcttcagtaa

SEQ ID NO: 30 - protein, 237 aa, Pyrococcus horikoshii
MTVVPRKGAVGIGTLIVFIAMVLVAAVAAAVLINTSGYLQQKASGTGRETTQEVASGIKVDRVVGYAPDITGDITRLAVYISPNAGSSGIDLNKVRVILSNGQKEVSLKYNYVYNATSSTQTYVALPQGNIFNDIVLGVNGTSENAASTQVNFNWSLLTGSTFGLIVLQDADGSVKASTPTLNQGDLVIIAIDVDAALGGIPPRTSITGEVIPEQGAPGVIEFTTPSTYTAHVMELQ

SEQ ID NO: 31 - DNA, 645 bp, Pyrococcus horikoshii
atgaggaagggagcaataggcattggtacactgatcgtctttatcgcaatggttctagtagccgcagtagccgcgggggtaatcataggaacagcaggttacctccagcagaaagcccaagcagcagggaggcaaacaacccaggaagttgcaagtggaataaagatcgtcaatgtattcggctacataaacgcaactcccccaagcaatggaacgatagtcaagatggccatcctggtaactcccaacgctgggagcagtggaattgacttaagcaacgttaagatagtgctcagcgatgggaagaggttagcggtttacaactacagcggagtactatacacggggaagatactcgacctcttcaacttgacgatctggaagaataccagcaacggaaccttcagcattgcagtggttaatgacgttggttcaaagatggagaaccaccacccaaccctcgagtggggtgacaccgttgcactgctcctcagaactgacgatgtcttcgagtacgaaggtaagggtggaatagggccatccacaaagataatagggaaggtgattccggatgctggagctgctggagttatagacttcacgactcccccaacgtttggctacaacgtgttagagttgcagtga

SEQ ID NO: 32 - protein, 214 aa, Pyrococcus horikoshii
MRKGAIGIGTLIVFIAMVLVAAVAAGVIIGTAGYLQQKAQAAGRQTTQEVASGIKIVNVFGYINATPPSNGTIVKMAILVTPNAGSSGIDLSNVKIVLSDGKRLAVYNYSGVLYTGKILDLFNLTIWKNTSNGTFSIAVVNDVGSKMENHHPTLEWGDTVALLLRTDDVFEYEGKGGIGPSTKIIGKVIPDAGAAGVIDFTTPPTFGYNVLELQ

SEQ ID NO: 33 - DNA, 924 bp, Sulfolobus solfataricus
atgaactccaaaaagatgttaaaggaatacaacaaaaaagtgaaaaggaaaggattagcgggattagacactgcaataatattaatagcatttataataactgcatcagtattagcttacgtggctataaatatgggattatttgtgacacagaaagccaaatccactataaataaaggagaggagacagcgtcaacagcactaacactatccggctctgtcctatatgctgttaactatccattaaatactagaagctactggatatactttacagtatctccaagttctggagtttctagcgtggaattgtcgcccactactacagccatctcgtttactgcatctgcagaaggagtgacgtactcaaatatatataaatacaccttattaacagtatccccatctgaactagcgaatgtcgtatacgcgaatggacagtacttagatctcgtaaatcagcagacaagtgcaggtcaaacatatgtatattatcctaatccttactatgcgttactagcacttaattacacactatataattattatcttagtacaaaaacaccatcaccaatatttattaatagtagcattctatctctatctagccttccatcatggttgaagaatgacaatagttttactttcactctcaatataagcggcaaactagttacttactatgtgtttgttaatcagacatttgcatttacatatccagtggcaggagatccgttaatagggagtgctatcgcccccgccggatcagtaataggagtaatacttttgtttggaccagatctaggaagtcatgtatttcaatatcagacaataacaatacaaattacaccaaatataggatctcctctcacaatatctgaatatatataccagccagagggtagcgtatcagtaatagggtga

SEQ ID NO: 34 - protein, 307 aa, Sulfolobus solfataricus
MNSKKMLKEYNKKVKRKGLAGLDTAIILIAFIITASVLAYVAINMGLFVTQKAKSTINKGEETASTALTLSGSVLYAVNYPLNTRSYWIYFTVSPSSGVSSVELSPTTTAISFTASAEGVTYSNIYKYTLLTVSPSELANVVYANGQYLDLVNQQTSAGQTYVYYPNPYYALLALNYTLYNYYLSTKTPSPIFINSSILSLSSLPSWLKNDNSFTFTLNISGKLVTYYVFVNQTFAFTYPVAGDPLIGSAIAPAGSVIGVILLFGPDLGSHVFQYQTITIQITPNIGSPLTISEYIYQPEGSVSVIG

SEQ ID NO: 35 - DNA, 924 bp, Sulfolobus solfataricus; identical to SEQ ID NO: 33

SEQ ID NO: 36 - protein, 307 aa, Sulfolobus solfataricus; identical to SEQ ID NO: 34

SEQ ID NO: 37 - DNA, 885 bp, Thermococcus kodakaraensis
atgaagaccagaacaaggaaaggtgcggttggtattggaaccctgattgttttcatagccatggttctagtggcggcagtggccgcggcagtgctgatcaacacgagcggctacctgcagcagaagagccaggctactggaagagagaccacccaggaagtagccagcggaataaaggtcgagagagtcgtcggtaagacagacctcccgtataccaacattggatccgattcaacggagcttgattacataaggcagctcgccatctacgtcagcccgaacgccggaagctcgggaatcgacctcagcaacaccaaggtcattctcagcaacggtgagaaggaggccgttctcaagtacgctggtggaccggatgatgattacgacgcattcaccaagggcgtccagaacgacatttttgacctgtactttaagtattcatcagatggtaccaactggaataatgagcacagtggtctcgccgcttggaagaacctctactacacgggtaccaaccacgacccggccaagaacttcggtatcatcgtcatccaggacgccgacaacagcctcaccgaagactacccgaccctcaacaagggcgacctcgtagtcctcacggtcctcgttggaagccttgaggagtacacaggtaatccttcaaatgacgacaatgctgtctacgaaactggtggcgccaagtacgactacattgacgttaatggcaatagcgatactactgataccatacagggcgtcttcggcgagggaatccccgccggtaccaagatcaccggtgaggtcgttccggagttcggcgctcctggcgtcatcgagttcaccaccccgagcacctacactgaggccgttatggagctccagtga

SEQ ID NO: 38 - protein, 294 aa, Thermococcus kodakaraensis
MKTRTRKGAVGIGTLIVFIAMVLVAAVAAAVLINTSGYLQQKSQATGRETTQEVASGIKVERVVGKTDLPYTNIGSDSTELDYIRQLAIYVSPNAGSSGIDLSNTKVILSNGEKEAVLKYAGGPDDDYDAFTKGVQNDIFDLYFKYSSDGTNWNNEHSGLAAWKNLYYTGTNHDPAKNFGIIVIQDADNSLTEDYPTLNKGDLVVLTVLVGSLEEYTGNPSNDDNAVYETGGAKYDYIDVNGNSDTTDTIQGVFGEGIPAGTKITGEVVPEFGAPGVIEFTTPSTYTEAVMELQ

SEQ ID NO: 39 - DNA, 777 bp, Thermococcus kodakaraensis
atgaggttccttaagaagcgtggtgcggttggtattggaactttgatagtgttcatcgccatggtgctcgttgcggcagttgccgcggcagtgctcatcaacaccagcggctacctccagcagaagagccagagcactggaaggcaaaccaccgaggaggtagccagcggaataaaggtaacgagcatcgttggctatgcaccatacgacgatagcaacaaggtgtacaagccaataagcaagcttgccatctacgtcagcccgaacgccggaagtgccggcatcgacatgaagaaggtcagggtaatactcagcgacggcagtatcgaggccgtgttgaagtatgacaattcggacgctgacagtgatggaacgcttgacaaagacgtcttcgccgtcggcatgcccgacaacgtgtttgaggatgacaccggcacaacggcctacgatggcgatcagtacatcacctggagcgaactcaacgacaagaccttcggcatcatagtcgtccaggacagcgacggctccctcaagccgctcaccccgaccctcaacaagggtgacatcgccataatcgccgtcagggttggcaattattacgttgacagcaacggtaacctccaggcatactcacccacaccagatggcgtcttcggcgaaggcatcaagcccaacacccacataaccggccaggtcgttccggagcacggtgcccctggcgtcattgacttcaccacaccgtcaacctatacccagagcgtcatggagctccagtga

SEQ ID NO: 40 - protein, 258 aa, Thermococcus kodakaraensis
MRFLKKRGAVGIGTLIVFIAMVLVAAVAAAVLINTSGYLQQKSQSTGRQTTEEVASGIKVTSIVGYAPYDDSNKVYKPISKLAIYVSPNAGSAGIDMKKVRVILSDGSIEAVLKYDNSDADSDGTLDKDVFAVGMPDNVFEDDTGTTAYDGDQYITWSELNDKTFGIIVVQDSDGSLKPLTPTLNKGDIAIIAVRVGNYYVDSNGNLQAYSPTPDGVFGEGIKPNTHITGQVVPEHGAPGVIDFTTPSTYTQSVMELQ

SEQ ID NO: 41 - DNA, 660 bp, Thermococcus kodakaraensis
atgcgtaggaggggagcaataggcatcggcacgctgatcgtcttcatcgcaatggtgctggttgctgcagtggccgcaggggtcatcatcggtacagcgggctaccttgagcagaaggctcaggccgctggcaggcagaccacacaggaggtagccagcggaataaaggtgctcaacgtctacggctacaccaacgccacacccccgagcaacggcacaatagagaggatggctatcttcataactcccaacgcaggcagtgagggcatcgacctgagcaacgttaagatagttctcagcgacggaaggaggctggtcgtttacaactactcgggtagcttccagaacgccgagagcgttaaggacctcttcaacatgacctacgttggcgtgtggaacagcacaaatggaacggccagctttggcatagccgtcatcaacgacataggcagcgagatgcagggaacccacccgacgcttgagttcggtgacatggtcgcgctatgcgtctggacgacgatgttcgagtacgaggataaggacggcataggcccgagcaccaggataaccggaaaggtcatccccgagaggggcgccgccggtgtgctcgacttcaccacgccggccacgttcagctacaacgtgatggtgctccagtga

SEQ ID NO: 42 - protein, 219 aa, Thermococcus kodakaraensis
MRRRGAIGIGTLIVFIAMVLVAAVAAGVIIGTAGYLEQKAQAAGRQTTQEVASGIKVLNVYGYTNATPPSNGTIERMAIFITPNAGSEGIDLSNVKIVLSDGRRLVVYNYSGSFQNAESVKDLFNMTYVGVWNSTNGTASFGIAVINDIGSEMQGTHPTLEFGDMVALCVWTTMFEYEDKDGIGPSTRITGKVIPERGAAGVLDFTTPATFSYNVMVLQ

SEQ ID NO: 43 - DNA, 828 bp, Thermococcus kodakaraensis
atgaggaggggagcaataggcattggaacgctcatcgtgttcattgccatggtgctggttgccgcggtggccgctggagtgctcataagcaccagcggctacctccagcagaaggcaatgagcgccggcaggcagaccacccaggaggtcgcgagcggaattaaggtgctcaacgtctacggctacatcaacggttcaacacccggcgcccacaatataaccagactcgtcctctacgtcagcccgaatgccggatccggtggcattgaccttgcccacgttaaggtcgtcataagcgacggcaagaggatggccgtttatcgttactacgaccccaatgaggacaaaaacagtgatatccagccagcttacatccactacacaggggacatcgctaacgtctttgcctatgagaagtgggagccgtactataaaggtaagtaccccaccgggtttgaccccaacaataagttctacataacggacaacatcgacataagcgccgtctggtggaacctctacagcgcctacaacaagaccagtaataatgataaggactacggtaaactcctctttggaattgcggtcgttcaggacggtgacgagagccttgacagtgagaaccaccccagcctcagctggggtgacatagcggccattatgctgtggacgttcccgtttgatgataacaacaatccgatcgatggattcggtctgccaccgagcaccaaggtcaccggaaaggtcatacctgagaacggtgcgggcggcgtcatagacttcacaacaccatcgacgtatactgacaacatactggaactccagtga

SEQ ID NO: 44 - protein, 275 aa, Thermococcus kodakaraensis
MRRGAIGIGTLIVFIAMVLVAAVAAGVLISTSGYLQQKAMSAGRQTTQEVASGIKVLNVYGYINGSTPGAHNITRLVLYVSPNAGSGGIDLAHVKVVISDGKRMAVYRYYDPNEDKNSDIQPAYIHYTGDIANVFAYEKWEPYYKGKYPTGFDPNNKFYITDNIDISAVWWNLYSAYNKTSNNDKDYGKLLFGIAVVQDGDESLDSENHPSLSWGDIAAIMLWTFPFDDNNNPIDGFGLPPSTKVTGKVIPENGAGGVIDFTTPSTYTDNILELQ

(19) United States
(12) Patent Application Publication
(10) Pub. No.: US 2008/0305524 A1
(43) Pub. Date: Dec. 11, 2008

(54) Molecular Glue

(75) Inventors: Reinhard Wirth, Regensburg (DE); Daniela Jasmin Nather, Hof (DE); Reinhard Rachel, Bad Abbach (DE); Gerhard Wanner, Moosburg (DE)

Correspondence Address: FULBRIGHT & JAWORSKI L.L.P., 600 CONGRESS AVE., SUITE 2400, AUSTIN, TX, 78701, US

(73) Assignees: UNIVERSITAT REGENSBURG, Regensburg (DE); LUDWIG-MAXIMILIANS-UNIVERSITAT, Munich (DE)

(21) Appl. No.: 11/915,840

(22) PCT Filed: May 30, 2006
(86) PCT No.: PCT/EP2006/005158

§ 371 Date: Jul. 7, 2008

(30) Foreign Application Priority Data
May 31, 2005 (EP) 05011704.3

(51) Int. Cl. C12P 21/00; C07K 14/44

(52) U.S. Cl. 435/69.1; 530/350; 530/412

(57) ABSTRACT

The present invention relates to an adhesive material being composed and/or consisting of at least one protein obtained or obtainable from flagella from archaea. Furthermore, the present invention relates to the use of at least one protein obtained from flagella from archaea for the method for the preparation of an adhesive material comprising the step of isolating and/or purifying at least one protein obtained from flagella from archaea.

The present invention relates to an adhesive material being composed and/or consisting of at least one protein obtained or obtainable from flagella from archaea. Furthermore, the present invention relates to the use of at least one protein obtained from flagella from archaea for the preparation of an adhesive material and a method for the preparation of an adhesive material comprising the step of isolating and/or purifying at least one protein obtained from flagella from archaea.

Surface organelles of prokaryotes used for motility are named flagella. In the case of eubacteria those organelles have been defined (at least for some species like the Enterobacteria Escherichia coli and Salmonella typhimurium) to a very high resolution, which is true for molecular and functional aspects. In the case of archaea (=archaebacteria) those organelles are defined only for a few restricted species like Halobacterium salinarum—e.g. Alam (1984; J. Mol. Biol. 176:459-475) and Tarasov (2000; Mol. Microbiol. 35:69-78) and Methanococcus voltae—e.g. Thomas (2002; Mol. Microbial. 46:879-887). In principle flagella of eubacteria and archaea differ in the following aspects:

| Bacterial flagella | Archaea flagella |
|---|---|
| composed of 1 flagellin | composed of several flagellins |
| flagellin very seldomly modified | flagellins very often glycosylated |
| flagellin without N-terminal | N-terminal leader peptide present in |
| leader peptide | flagellin |
| anchored via basal body, rings | no specific anchoring structure known |
| and hook | |
| diameter ca. 20 nm with central | diameter 10-15 nm without central |
| channel of ca. 2 nm | channel |
| used for swimming | used for swimming |

The statements given above refer to the rule, but—as usual—some exceptions exist. It is evident that archaea flagella are much less characterized than their bacterial counterparts; nearly nothing is known about their assembly. It is argued—but not proven—that a totally different assembly process is used by archaea, compared with bacteria. Whilst the former seem to build the surface appendage from the basis (=addition of monomers at the cytoplasmic membrane) it has been very clearly proven that synthesis is from the tip of the organelle in the case of bacteria.

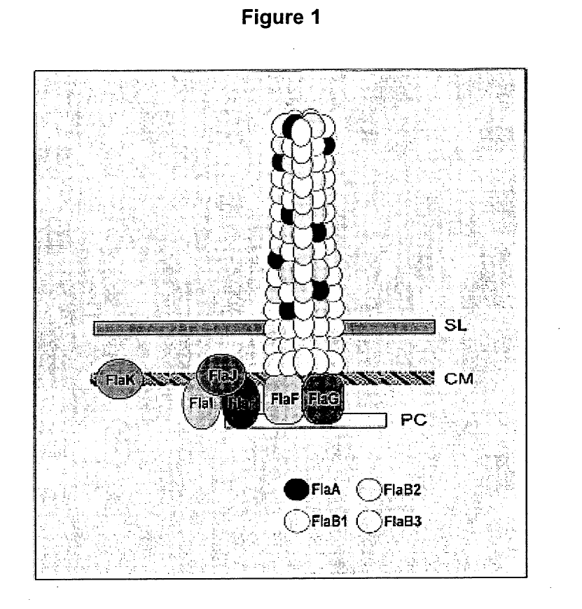

Flagellins are defined as proteins constituting the long filaments; in the case of archaea many of those are glycosylated. It has to be noted that proteins not directly forming the filaments, but being associated with the cytoplasmic membrane and being needed for the correct processing, transport, modification and assembly of the filament monomers sometimes also are called “Fla-proteins” (see FIG. 1).

A model for subcellular location of different Fla proteins of archaea was proposed by Bardy (2003; Microbiology 149:295-304). In this case the flagellum of M. voltae is composed of three different flagellins, namely FlaA, FlaB1 and FlaB2. The location of FlaB3 at the base of the structure has been deduced indirectly, whilst no function could be assigned to the membrane-associated proteins Flaf, FlaG, FlaH, FlaI and FlaJ yet. FlaK is a signal peptidase removing a signal peptide which can be very short (only 4 amino acids in some Fla proteins from different Pyrococci), and in most cases is 12 amino acids long.

Few functional studies for archaea flagella have been published. In the case of H. salinarum Marwan (1991; J. Bacteriol. 173:1971-1977) could show that the surface organelle can rotate in clockwise and counterclockwise direction and therefore the mode of motion in principal corresponds to that of eubacteria. In the case of M. voltae which is studied most intensively by K. Jarrell's group such a rotation has—to the best of our knowledge—not been demonstrated yet.

The problem underlying the present invention is the development of a glue which is heatstable and/or which can also be applied in wet and moist environments. Presently used glues are often epoxy based, cement based or based on synthetic polymers. Both the epoxy compounds and the synthetic polymers may leach and constitute a risk to the environment. Their application often requires mechanical working or kneading of the glue or sealing agent, in order to remove the water present on the surfaces. There is a need for new glues, better adapted for use in warm and/or moist environments or for underwater use and more environmentally friendly than the present products.

Furthermore, there is a need for the provision of materials and compositions which may be efficiently employed as “glue” in (nano)technology applications, like the preparation of chips, in particular DNA chips/arrays or protein chips/arrays, like antibody arrays.

The solution to said technical problem is achieved by the embodiments provided herein and as characterized in the claims. Accordingly, the present invention relates to an adhesive material being composed and/or consisting of at least one protein obtained or obtainable from flagella from archaea.

Before the present invention is described in detail, it is to be understood that this invention is not limited to the particular methodology, protocols, bacteria, vectors, and reagents etc. described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W., Nagel, B. and Kölbl, H. eds. (1995), Helvetica Chimica Acta, CH-4010 Basel, Switzerland). Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

It must be noted that as used herein and in the appended claims, the singular forms “a”, “an”, and “the”, include plural referents unless the context clearly indicates otherwise. Thus, for example, reference to “a reagent” includes one or more of such different reagents, and reference to “the method” includes reference to equivalent steps and methods known to those of ordinary skill in the art that could be modified or substituted for the methods described herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

The domain archaea (=archaebacteria) comprises according to the Systema Naturae 2000 (http://sn2000.taxonomy.nl) the phyla Crenarchaeota, Euryarchaeota and Nanoarchaeota. These phyla include further classes which are known to the skilled person. Among the phyla are, for example halophiles or thermophiles.

By using classical systematics, for example, by reference to the pertinent descriptions in “Bergey's Manual of Systematic Bacteriology” (Williams & Wilkins Co., 1984), the skilled person can determine whether a prokaryote is an archaeum. Alternatively, the affiliation of a prokaryote to the archaea can be characterized with regard to ribosomal RNA in a so called Riboprinter®. More preferably, the affiliation of a prokaryote to the archaea is demonstrated by comparing the nucleotide sequence of the 16S ribosomal RNA of such a prokaryote, or of its genomic DNA which codes for the 16S ribosomal RNA, with those of other known archaea. Another alternative for determining the affiliation of a prokaryote to the archaea is the use of species- or domain-specific PCR primers that target the 16S-23S rRNA spacer region.

In accordance with the present invention, it was surprisingly found that specific surface organelles of archaea, in particular of (hyper)thermophilic archaea are not only used for motility of said archaea (e.g. swimming) but also contribute significantly to the adhesion on solid surfaces as described herein, in particular to metal surfaces, quartz/silica surfaces and the like.

Accordingly, the present study relates in particular to surface organelles and in particular to isolated proteins/proteinaceous structures of archaea, preferably (hyper)thermophilic archaea, especially of P. furiosus.

The term “isolated” means that the material is removed from its original environment, e.g. the natural environment if it is naturally-occurring. For example, a naturally-occurring protein not isolated, but the same protein, separated from some or all of the coexisting materials in the natural system, is isolated.

Of course, also archaea being (extreme) halophiles, alkalophiles, or acidophiles might be the source of those proteins/structures. The archaeum P. furiosus was named by Fiala (1986; Arch. Microbiol. 145:56-61) a rushing fireball because of the ability of the coccoid cells to rapidly swim at the optimal growth temperatures between 90 to 100° C. The studies were carried out in a thermomicroscope allowing studies of swimming behaviour up to 95° C. under strictly anaerobic conditions. The biochemical analysis of P. furiosus flagella provided herein demonstrated that they are composed of only one flagellin. In addition it was successfully observed that the flagella can aggregate into cable-like structures connecting cells. It could also be shown that flagella enable the archaeal cells to adhere to surfaces, allowing growth in a biofilm-like manner. Therefore these surface appendages which are assumed to function in motility are multifunctional organelles.

Since most other archaea face the same problem like P. furiosus namely to steadily stay in very sharply defined regions in their natural habitat (otherwise they would be swamped away form places having e.g. temperatures they need for growth) the present invention also provides for the fact that other flagellar proteins of archaea have a similar adhesion function. The advantage of using adhesins of archaea over those from eubacteria as “molecular glue” lies in the fact that in many cases these proteins are optimised for extreme conditions—in the case of P. furiosus e.g. to temperatures between 0-100° C., 10-100° C., 20-100° C., 30-100° C., 40-100° C., 50-100° C., 60-100° C., 70-100° C., 80-100° C., 90-100° or around or above 100° C.

The applications for such a protein glue are seen in the field of nano(bio)technology. Proteins acting as molecular cement to connect part A to part B do not have the disadvantage of chemicals which might interfere with the biological functions of one of the parts. Quantum dots e.g. have to be functionalised by a shell of polyacrylic acid to allow conjugation to macromolecules and ligands. Archaeal Fla (flagellin) proteins—optimised e.g. for low pH (e.g. Sulfolobus), high salt (e.g. Halobacterium), high pH (e.g. Natrialba), high temperature (many hyperthermophilic archaea, like Thermococcus or Pyrococcus)—are attractive adhesive materials to be employed in a variety of uses, like in medical settings, as well as in technologies like the use in the preparation of protein chips or nucleic acid molecule chips. Also the use in nanotechnology is envisaged.

Halophilic archaea are divided into slightly halophilic having optimal growth at 1-5% (w/v) NaCl, moderately halophilic with optimal growth at 5-18% (w/v) NaCl and extremely halophilic with optimal growth above 18% (w/v) NaCl. Also in the present context, halotolerant archaea are defined as microorganisms selected from the following types: slightly halotolerant which grow at NaCl concentrations up to 6-8% (w/v) NaCl, moderately halotolerant which grow at NaCl concentrations up to 18-20% (w/v) NaCl, and extremely halotolerant growing at NaCl concentrations up to and above 20% (w/v) and occasionally to the point of saturation of NaCl (approx. 36% (w/v) NaCl).

Acidophilic archaea can be divided into moderate acidophilic archaea growing above pH 4, acidophilic archaea growing between pH 1.5 to 4 and extreme acidophilic archaea growing between pH 0 to 2. Alkaliphilic archaea can be divided into moderate alkaliphilic ones growing up to pH 9, into alkaliphilic ones growing best at pH 8.5 to 10; extreme alkaliphilic archaea do possess ph optima for growth of 11 or even higher.

Since archaeal flagellins in most cases are glycoproteins the question arises how one can obtain sufficient amounts for (nano)technological as well as medical applications.

A first alternative is the isolation of the material directly from cells after fermentation. A main advantage of using P. furiosus as starting material for a molecular glue is its rapid growth with doubling times of 35 min at 95° C.; i.e. a 300 l fermentor grows up overnight.

A second alternative is the use of eukaryotic cells—like CHO cells or insect cells—and expression vectors developed for them for production of recombinant proteins. Potential insect systems would be e.g. the DES-system (Drosophila expression system by Invitrogen) or the Sf9/Sf21 system (ovarian cells from the butterfly Spodoptera frugiperda).

A further alternative is the expression of archaeal flagellins in bacteria, especially in Escherichia coli. It has to be noted that Bayley (1999; J. Bacteriol. 181:4146-4153) was successful in this respect with structural proteins from M. voltae flagella, and therefore such an approach is reproducible by the skilled artisan. In this case, expression of flaB2 was possible in E. coli using pT7-7 (a T7 promoter based expression vector) and in Pseudomonas aeruginosa using pUCP18/19, an E. coli-P. aeruginosa shuttle vector.

Another alternative is the use of a yeast expression system as described in WO 02/00879. In particular said PCT-application describes host cells derived from unicellular or filamentous fungi which are lacking a key enzyme of yeast glycosylation. Accordingly, said host cells are not capable of glycosylating proteins in a yeast-like manner leading to high-mannose structures. Thus, after transforming said modified host cells with enzymes involved in glycosylation processes in archaea, it could be envisaged that a desired archaeal protein is produced by yeast having a glycosylation pattern as occurring in its natural host. Very recently Voisin (2005; J. Biol. Chem. 280:16586-16593) was able to determine the glycosylation pattern of M. voltae flagellins using microtechniques. Accordingly, it is expected that glycosylating enzymes of archaea can be identified and isolated and, thus, can be used for the aforementioned purpose when expressing an archaeal protein in an artificial yeast expression system.

A further alternative that could be envisaged is to express the genes directly in archaea. Expression systems for archaea are known in the art and described, for example, in WO 2004/106527.

Fla proteins (flagellins) purified from sheared flagella or expressed in recombinant form may be applied onto various surfaces, like e.g. metals such as gold, copper, nickel, silicon, quartz, alumina, silica, for example, in the form of wafers, polymers such as plastic polymers, for example, polyvinylchloride, polycarbonate, nylon, wood etc. A test system for the “adhesive capacity” of a given archaeal flagellin is provided as follows: After a certain binding time to surfaces, these are washed thoroughly and tested for adherence of the Fla proteins. Detection of bound Fla proteins might be via immunological or by direct staining methods. In the first case antibodies against purified Fla proteins are applied onto the surfaces and after a certain binding time unbound antibodies are removed by washing steps. Antibodies bound to Fla proteins which themselves adhere to the surfaces can be detected via various available techniques including secondary antibodies coupled to enzymes resulting in colour development, resulting in chemiluminescence, etc. In the second case one might label bound Fla proteins with fluorescent dyes like e.g. AlexaFluor succinimidyl esters.

The person skilled in the art can easily obtain archaeal flagellins by methods known in the art and by methods provided herein. In accordance with this invention, the term “flagellin” is synonymous with the term “Fla proteins”. As is evident from the appended experimental part of this invention, the “flagellin” to be employed in context of this invention relates to proteins derived from the non-membrane associated part of the “flagella” of archaea. Said flagellins are proteins constituting the long filaments of archaea. Flagellins may be glycosylated. Accordingly, for example the flagellins to be employed in context of this invention relate, inter alia, to the flagellin proteins FlaA, FlaB₁ and FlaB₂ of M. vannielli, to FlaB of P. furiosus, to FlaB₁ of H. sp. NRC1, to FlaB₂ of H. sp. NRC1, to the subunit (1) of N. magadii, to the subunit (2) of N. magadii, to the subunit (3) of N. magadii, to the subunit (4) of N. magadii, to FlaB1-1_b5 of P. abysii, to FlaB1-2_b4 of P. abysii, to FlaB1-3_b2 of P. abysii, to 1, 2, 3, 4, or 5 of P. horikoshii, to 1 or 2 of S. solfataricus, to B1, B3, B4 or B5 of T. kodakaraensis. Corresponding amino acid sequences are illustratively given in the appendix as “flagellin sequences from archaea” and are also shown in the sequence listing. The person skilled in the art realizes from the present invention that naturally membrane-associated flagellins (in FIG. 1 SL stands for surface layer, CM stands for cytoplasmic membrane and PC stands for polar cap) are not comprised in the gist of the present invention. Such membrane-associated flagellins are, without being bound by theory, associated with the cytoplasmic membrane and needed for the correct processing, transport, modification and assembly of the filament monomers.

Archaeal flagellins comprise, but are not limited to the archaeal flagellins shown in SEQ ID NOs: 2, 4, 6, 8, 10, 12, 14, 16, 18, 20, 22, 24, 26, 28, 30, 32, 34, 36, 38, 40, 42 or 44 or as encoded by nucleic acid molecules as shown in any one of SEQ ID NOs: 1, 3, 5, 7, 9, 11, 13, 15, 17, 19, 21, 23, 25, 27, 29, 31, 33, 35, 37, 39, 41 or 43.

A particular preferred flagellin in context of this invention is the single flagellin obtainable from P. furiosus, in particular from P. furiosus as deposited under DSM3638. The corresponding Fla proteins/flagellins are of particular use in the preparation of the adhesive material(s)/glue(s) as disclosed herein.

The present inventors now make available a characterised and purified flagellin with many uses in medicine and other technical applications as disclosed in the following description, examples and claims.

The present invention makes available a substantially pure adhesive protein, namely archaeal flagellins, comprising preferably, but not limited to, the amino acid sequences as shown in any one of SEQ ID Nos. 2, 4, 6, 8, 10, 12, 14, 16, 18, 20, 22, 24, 26, 28, 30, 32, 34, 36, 38, 40, 42 or 44 (or fragments thereof), including functionally equivalent fragments or variants thereof.

The term “substantially pure”, as used herein, means a protein that has been separated from other proteins, lipids, and nucleic acids with which it naturally occurs. Preferably, the protein is also separated from other substances, e.g., antibodies, matrices, etc., which may be used to purify it.

Since archaeal flagellins are conserved more or less over about preferably the first 30, 35, 40, 45 or 50 amino acids it is assumed that this part of the protein is responsible for a common function of all flagellins like e.g. self assembly. The term “functionally equivalent” is meant to encompass proteins, or polynucleotide sequences exhibiting equivalent properties with respect to any desired quality in question, such as the adhesive property and/or the potential to form adhesive films. Fragments of the proteins of the invention comprise preferably at least 30, 35, 40, 45, 50, 55, 60 amino acids of amino acid sequences as shown in any one of SEQ ID Nos. 2, 4, 6, 8, 10, 12, 14, 16, 18, 20, 22, 24, 26, 28, 30, 32, 34, 36, 38, 40, 42 or 44.

A “variant” or “derivative” of a protein of the present invention encompasses a protein, wherein one or more, preferably one to 50, one to 40, one to 30, one to 20, one to 10 or one to 5 amino acid residues are substituted, preferably conservatively substituted compared to said protein and wherein said variant or derivative exhibits preferably adhesive property and/or the potential to form adhesive films.

Such variants or derivatives include deletions, insertions, inversions, repeats, and substitutions selected according to general rules known in the art so as have no effect on the activity of a polypeptide of the present invention. Accordingly, it is envisaged that one or more, preferably one to 50, one to 40, one to 30, one to 20, one to 10 or one to 5 amino acid residues are deleted or inserted.

For example, guidance concerning how to make phenotypically silent amino acid substitutions is provided in Bowie, Science 247: (1990) 1306-1310, wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein. The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244: (1989) 1081-1085.) The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved.

The invention encompasses polypeptides having a lower degree of identity but having sufficient similarity so as to perform one or more of the functions performed by a polypeptide as described herein. Similarity is determined by conserved amino acid substitution. Such substitutions are those that substitute a given amino acid in a polypeptide by another amino acid of like characteristics (e.g., chemical properties). According to Cunningham et al. above, such conservative substitutions are likely to be phenotypically silent. Additional guidance concerning which amino acid changes are likely to be phenotypically silent is found in Bowie, Science 247: (1990) 1306-1310.

Tolerated conservative amino acid substitutions of the present invention involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and H is; replacement of the aromatic residues Phe, Tyr, and Trp, and replacement of the small-sized amino acids Ala, Ser, Thr, Met, and Gly.

In addition, the present invention also encompasses the conservative substitutions provided in the Table below.

| For Amino Acid | Code | Replace with any of: |
|---|---|---|
| Alanine | A | D-Ala, Gly, beta-Ala, L-Cys, D-Cys |
| Arginine | R | D-Arg, Lys, D-Lys, homo-Arg, D-homo- |
| | | Arg, |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-As |
| Glutamic Acid | E | D-Glu, D-Asp, Asp, Asn, D-Asn, Gln, D- |
| Glycine | G | Ala, D-Ala, Pro, D-Pro, β-Ala, Acp |
| Isoleucine | | D-Ile, Val, D-Val, Leu, D-Leu, Met, D-Met |
| Leucine | L | D-Leu, Val, D-Val, Met, D-Met |
| Lysine | K | D-Lys, Arg, D-Arg, homo-Arg, D-homo- |
| | | Arg, |
| Methionine | M | D-Met, S-Me-Cys, Ile, D-Ile, Leu, D-Leu, |
| | | Val, |
| Phenylalanine | F | D-Phe, Tyr, D-Thr, L-Dopa, His, D-His, Trp, |
| | | D-Trp, Trans-3,4, or 5-phenylproline, cis- |
| | | 3,4, or 5-phenylproline |
| Proline | P | D-Pro, L-1-thioazolidine-4-carboxylic acid, D- |
| | | or L-1-oxazolidine-4-carboxylic acid |
| Serine | S | D-Ser, Thr, D-Thr, allo-Thr, Met, D-Met, |
| | | Met(O), D-Met(0), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Aside from the uses described above, such amino acid substitutions may also increase protein or peptide stability. The invention encompasses amino acid substitutions that contain, for example, one or more non-peptide bonds (which replace the peptide bonds) in the protein or peptide sequence. Also included are substitutions that include amino acid residues other than naturally occurring L-amino acids, e.g., D-amino acids or non-naturally occurring or synthetic amino acids, e.g., 1 or γ amino acids.

Both identity and similarity can be readily calculated by reference to the following publications: Computational Molecular Biology, Lesk, A. M., ed., Oxford University Press, New York, 1988; Biocomputing: Informatics and Genome Projects, Smith, DM., ed., Academic Press, New York, 1993; Informatics Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, eds., M Stockton Press, New York, 1991.

The adhesive property of the archaeal flagellins, in particular the flagellin obtainable form P. furiosus, as described herein is particularly useful in medical as well as in technological settings.

It is of note that in the uses provided herein, it is also envisaged that a mixture of flagellin proteins (e.g. a mixture of adhesive flagellins derived and/or obtainable from different species) be employed. Accordingly, the term “at least one flagellin” as used herein also means that mixtures of adhesive flagellins (derived from archaeal flagella) be used in the preparation of the inventive adhesive material/glue.

For example, the adhesive flagellin may be used in medical applications, for example as a component in wound dressings and bandages, in particular in such applications where the biodegradable properties of the protein are needed. It is also envisaged that the adhesive property of archaeal flagellins be employed in the coating of (medical) bands and strings. Since the archaeal flagellins as documented herein have an ability to attach to surfaces, and to form an attachment between surfaces, they may be used as a tissue adhesive. The adhesive capability of flagellin (Fla protein) may, accordingly, be used as an adhesive for plasters, adhesives, bandages, patches and dressings etc. The protein may also be useful in orthopaedics as a glue to keep or hold joint replacements together. It is also envisaged to use the adhesive properties of the flagellins as surface coating of medical and/or surgical devices and tools, e.g. stents, chirurgical nails, suture, implants or transplants. The use of the adhesive flagellins derived from archaeal flagella in dental medicine is also envisaged, for example in the anchorage/attachment of artificial tooth parts or crowns. Furthermore, the use of the adhesive flagellins as provided herein in dental restoration or for dental implants is envisaged.

One embodiment of the present invention is the application of the Fla proteins (flagellins) as such, derivatives thereof or information derived thereof for the production of a glue or an adhesive for use in moist environments. Moist environments in this context include both aquatic environments, objects and surfaces in contact with water, sea water, fresh water, high humidity, steam and/or condensation. The applications can be found in both natural or man-made environments and even on or within an animal or human body.

Since the flagellins to be employed in accordance with this invention are obtained from or derived from archaea cells which need extreme environmental conditions for growth, like high salt, low pH and/or high temperature, the “molecular adhesives/glues” provided in this invention are particular useful in extreme conditions, like high salt concentrations or in high temperature applications. This fact makes the herein provided uses of archaeal flagellins as molecular glue(s) attractive in (nano)technological applications.

The present invention provides for the use of at least one protein obtained from flagella from archaea for the preparation of an adhesive material. As documented herein and as illustrated in the appended examples, said at least one protein is more preferably a flagellin from archaea, and most preferably a flagellin from P. furiosus.

As detailed below, also a method for the preparation of an adhesive material or a glue comprising the step of isolating and/or purifying at least one protein obtained from flagella from archaea, namely a flagellin, is provided in context of this invention.

As discussed above “said at least one protein obtained from flagella from archaea” is preferably recombinantly produced, chemically isolated from flagella or chemically synthesized. Recombinant methods for the preparation comprise, but are not limited to, amplification of the coding region (including the signal peptide) via PCR (introducing special restriction sites), cloning into the E. coli vector pT7-7, transformation of the resulting construct into E. coli BL21(DE3)/pLysS, expressing the protein in the recombinant strain by induction with IPTG (isopropylthio-β-D-galactoside), harvesting the cells prior to lysis, separation of cellular proteins—including the recombinant flagellin—via SDS-PAGE, and excising the flagellin from the gel. Expression to a low level also can be in e.g. Pseudomonas aeruginosa PAK using pUCP18 as vector. In the E. coli system introduction of e.g. hexa-Histidin-tags can aid in purification of the recombinant protein; the signal peptide sequence not necessarily has to be present in the construct (own unpublished results).

It may also be possible to apply the IMPACT systems provided by New England Biolabs (NEB).

When applying expression systems which are, for example, commercially available, the skilled person knows that sometimes modifications of, for example, the manufacturer's instructions have to be made so as to customize the expression system for the protein desired to be expressed.

In addition, the skilled person when aiming at the recombinant expression of any of the proteins of the present invention may co-express, for example, one or more chaperons or chaperon-like proteins which may facilitate and/or enhance expression. In context of this invention, the at least one protein obtained from flagella from archaea is preferably a flagellin.

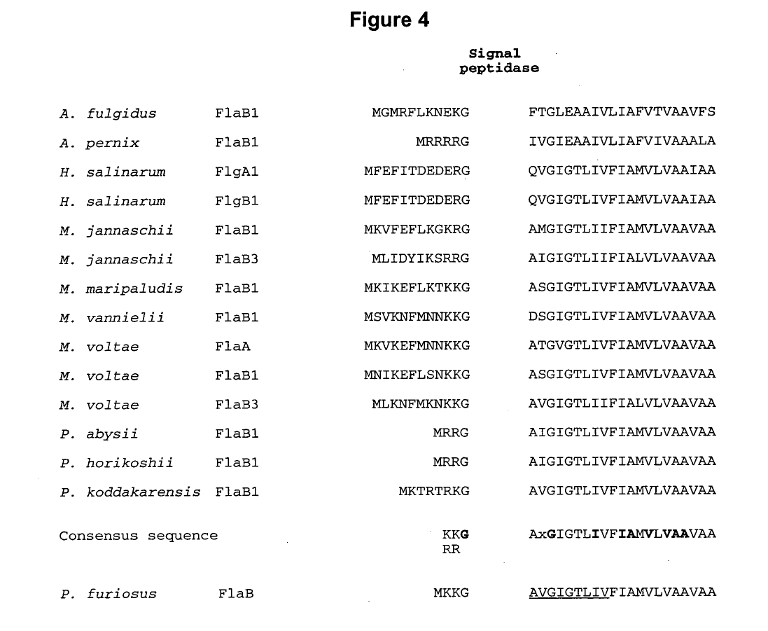

Most preferably said flagellin is a flagellin obtained and/or derived from A. fulgidus, A. pemix, H. salinarum, M. jannaschii, M. maripaludis, M. vannielii, M. voltae, P. abysii, P. horikoshii, P. kodakarensis, P. furiosus, (see also FIG. 4).

In a particular preferred embodiment of the adhesive material, the use or the method of the present invention said flagellin is encoded by a polynucleotide selected from the group consisting of
(a) a polynucleotide having a nucleotide sequence encoding the polypeptide having the deduced amino acid sequence as shown in SEQ ID NOs: 2, 4, 6, 8, 10, 12, 14, 16, 18, 20, 22, 24, 26, 28, 30, 32, 34, 36, 38, 40, 42 or 44;
(b) a polynucleotide having the coding sequence as shown in SEQ ID NOs: 1, 3, 5, 7, 9, 11, 13, 15, 17, 19, 21, 23, 25, 27, 29, 31, 33, 35, 37, 39, 41 or 43;
(c) a polynucleotide having a nucleotide sequence encoding a fragment or derivative of a polypeptide encoded by a polynucleotide of any one of (a) or (b), wherein in said derivative one or more amino acid residues are conservatively substituted compared to said polypeptide, and said fragment or derivative encodes an archaeal flagellin;
(d) a polynucleotide having a nucleotide sequence which is at least 70% identical to a polynucleotide as defined in any one of (a) to (c) and which encodes an archaeal flagellin;
(e) a polynucleotide having a nucleotide sequence the complementary strand of which hybridizes to a polynucleotide as defined in any one of (a) to (d) and which encodes an archaeal flagellin; and
(f) a polynucleotide having a nucleotide sequence being degenerate to the nucleotide sequence of the polynucleotide of any one of (a) to (e); or the complementary strand of such a polynucleotide.

The term “having” when used herein in the context of nucleotide sequences or amino acid sequences is interchangeable with the term “comprising”. The term “archaea or archaeal flagellin” as used in context of this invention is characterized in being a functional flagellin (or a functional fragment or derivative thereof capable of adhering to surfaces and/or surface like structures (like grids), whereby said surfaces and surface like structure may in particular be of inorganic material, like metals, plastics and the like. Said flagellin is derived from or naturally occurring in the “flagella” and is, naturally, not membrane associated. It is also envisaged to cover/coat materials like carbon fibers, glass fibers, textile filaments, plastic filaments and the like with the flagellin described herein. Also envisaged is the coating of porous material, like sponges and silica (e.g. silicium oxide) with the adhesive flagellin protein.

The adhesive flagellins may, accordingly, be employed to bind, stabilize and/or adhere secondary materials to primary materials. Illustratively, such a secondary material may be (without limitation) pigments, microparticles, catalyst particles, filler particles, polyelectrolyte capsules, colloidal particles, proteinaceous structures, nucleic acid molecules, and the like. Corresponding primary material may, non-limiting, be metals, silicon, alumina, silica, plastics or other oxides, polymers, fiber material (like carbon or glass fibers) and textile fibers. The adhesive flagellins may, accordingly, also be employed to bind, stabilize and/or adhere secondary materials to primary materials, both materials being characterized mainly by a large structural difference, e.g. secondary material being a foam, non-woven, textile material or aerogel and the primary material being, non limiting, a bulk solid, sheet material or thin.

In accordance with the present invention, the term “polynucleotide” means the sequence of bases comprising purine- and pyrimidine bases which are comprised by nucleic acid molecules, whereby said bases represent the primary structure of a nucleic acid molecule. Nucleic acid sequences include DNA, cDNA, genomic DNA, RNA, synthetic forms and mixed polymers, both sense and antisense strands, or may contain non-natural or derivatized nucleotide bases, as will be readily appreciated by those skilled in the art.

When used in accordance with the present invention the term “being degenerate” means that due to the redundancy of the genetic code different nucleotide sequences code for the same amino acid.

Of course, the present invention also envisages the complementary strand to the aforementioned and below mentioned nucleic acid molecules if they may be in a single-stranded form.

When used herein, the term “polypeptide” means (a) peptide(s), (a) protein(s), or (a) polypeptide(s) which encompasses amino acid chains of a given length, wherein the amino acid residues are linked by covalent peptide bonds. The term “polypeptide” when used herein is understood to be interchangeable with the term “protein”. However, peptidomimetics of such proteins/polypeptides wherein amino acid(s) and/or peptide bond(s) have been replaced by functional analogs are also encompassed by the invention as well as other than the 20 gene-encoded amino acids, such as e.g. selenocysteine or pyrrolysine. Peptides, oligopeptides and proteins may be termed polypeptides. The terms polypeptide and protein are often used interchangeably herein. It will be appreciated that polypeptides often contain amino acids other than the 20 amino acids commonly referred to as the 20 naturally occurring amino acids, and that many amino acids, including the terminal amino acids, may be modified in a given polypeptide, either by natural processes, such as processing and other post-translational modifications, but also by chemical modification techniques which are well known to the art. Even the common modification that occur naturally in polypeptides are too numerous to list exhaustively here, but they are well described in basic texts and in more detailed monographs, as well as in a voluminous research literature, and they are well known to those of skill in the art.

The basic structure of polypeptides and the recombinant or synthetic production as well as isolation methods of polypeptides are well known and have been described in innumerable textbooks and other publications in the art.

The polypeptides of the present invention are shown in SEQ ID NOs.: 2, 4, 6, 8, 10, 12, 14, 16, 18, 20, 22, 24, 26, 28, 30, 32, 34, 36, 38, 40, 42 or 44. They comprise, inter alia, the consensus motif AxGIGTLIVFIAMVLVAAVAA as described herein. “x” can be any amino acid. Said polypeptides may, e.g., be a naturally purified product as described herein or a product of chemical synthetic procedures or produced by recombinant techniques from a prokaryotic or eukaroytic host (for example, by bacterial, yeast, insect, mammalian cells in culture or plant cells in culture and/or as is known in the art).

Depending upon the host employed in a recombinant production procedure, the polypeptide of the present invention may be glycosylated or may be non-glycosylated. The polypeptide of the invention may also include an initial methionine amino acid residue. The polypeptide according to the invention may be further modified to contain additional chemical moieties not normally part of the polypeptide as described herein above. Those derivatized moieties may, e.g., improve the stability, solubility, the biological half life or absorption of the polypeptide. The moieties may also reduce or eliminate any undesirable side effects of the polypeptide and the like. An overview for these moieties can be found, e.g., in Remington's Pharmaceutical Sciences (18^{th} ed., Mack Publishing Co., Easton, Pa. (1990)). Polyethylene glycol (PEG) is an example for such a chemical moiety which has been used for the preparation of therapeutic polypeptides. The attachment of PEG to polypeptides has been shown to protect them against proteolysis (Sada, J. Fermentation Bioengineering 71 (1991), 137-139). Various methods are available for the attachment of certain PEG moieties to polypeptides (for review see: Abuchowski, in “Enzymes as Drugs”; Holcerberg and Roberts, eds. (1981), 367-383). Generally, PEG molecules are connected to the polypeptide via a reactive group found on the polypeptide. Amino groups, e.g. on lysines or the amino terminus of the polypeptide are convenient for this attachment among others.

The present invention also relates to the polynucleotides which encode a polypeptide, which has a homology, that is to say a sequence identity, of at least 30%, preferably of at least 40%, more preferably of at least 50%, even more preferably of at least 60% and particularly preferred of at least 70%, especially preferred of at least 80% and even more preferred of at least 85%, 90%, 95%, 96%, 97%, 98% or 99% to the amino acid sequence as shown in SEQ ID NOs.: 2, 4, 6, 8, 10, 12, 14, 16, 18, 20, 22, 24, 26, 28, 30, 32, 34, 36, 38, 40, 42 or 44. Such homologs of the polypeptide of the present invention encode a flagellin which is preferably useful as an adhesive material.

In order to determine whether a nucleic acid sequence or an amino acid sequence has a certain degree of identity to the nucleic acid sequence encoding a flagellin or to an amino acid sequence of a flagellin, the skilled person can use means and methods well-known in the art, e.g., alignments, either manually or by using computer programs such as those mentioned further down below in connection with the definition of the term “hybridization” and degrees of homology.

For example, BLAST2.0, which stands for Basic Local Alignment Search Tool (Altschul, Nucl. Acids Res. 25 (1997), 3389-3402; Altschul, J. Mol. Evol. 36 (1993), 290-300; Altschul, J. Mol. Biol. 215 (1990), 403-410), can be used to search for local sequence alignments. BLAST produces alignments of both nucleotide and amino acid sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cutoff score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST (Altschul (1997), loc. cit.; Altschul (1993), loc. cit.; Altschul (1990), loc. cit.) are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

$$\frac{\% sequence identity \times \% maximum B L A S T score}{100}$$

and it takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules.

The present invention also relates to nucleic acid molecules which hybridize to one of the above described nucleic acid molecules and which encode a flagellin.

The term “hybridizes” as used in accordance with the present invention may relate to hybridization under stringent or non-stringent conditions. If not further specified, the conditions are preferably non-stringent. Said hybridization conditions may be established according to conventional protocols described, for example, in Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989), or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as 0.1×SSC, 0.1% SDS at 65° C. Non-stringent hybridization conditions for the detection of homologous or not exactly complementary sequences may be set at 6×SSC, 1% SDS at 65° C. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. Such fragments may represent nucleic acid sequences which encode a flagellin, and which have a length of at least 12 nucleotides, preferably at least 15, more preferably at least 18, more preferably of at least 21 nucleotides, more preferably at least 30 nucleotides, even more preferably at least 40 nucleotides and most preferably at least 60, 70, 80, 90, 100 or 150 nucleotides. Furthermore, nucleic acid molecules which hybridize with any of the aforementioned nucleic acid molecules also include complementary fragments, derivatives and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The terms complementary or complementarity refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules. The degree of complementarity between nucleic acid strands has significant effects on the efficiency and strength of hybridization between nucleic acid strands. This is of particular importance in amplification reactions, which depend upon binding between nucleic acids strands.

The term “hybridizing sequences” preferably refers to sequences which display a sequence identity of at least 40%, preferably at least 50%, more preferably at least 60%, even more preferably at least 70%, particularly preferred at least 80%, more particularly preferred at least 90%, even more particularly preferred at least 95%, 97% or 98% and most preferably at least 99% identity with a nucleic acid sequence as described above encoding a flagellin to be employed in context of this invention, in particular as molecular glue. Moreover, the term “hybridizing sequences” refers to sequences encoding a flagellin having a sequence identity of at least 40%, preferably at least 50%, more preferably at least 60%, even more preferably at least 70%, particularly preferred at least 80%, more particularly preferred at least 90%, even more particularly preferred at least 95%, 97% or 98% and most preferably at least 99% identity with an amino acid sequence of a flagellin as described herein above. In accordance with the present invention, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95%, 97%, 98% or 99% identity), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson, Nucl. Acids Res. 2 (1994), 4673-4680) or FASTDB (Brutlag, Comp. App. Biosci. 6 (1990), 237-245), as known in the art.

Polynucleotides which hybridize with the polynucleotides of the invention can, in principle, encode a flagellin or can encode modified versions thereof.

Polynucleotides which hybridize with the polynucleotides disclosed in connection with the invention can for instance be isolated from genomic libraries or cDNA libraries of archaeas having a flagellin of interest. Preferably, such polynucleotides are from archaeal origin.

The polynucleotide of the invention may also be a variant, analog or paralog of such a polynucleotide as described herein. As used herein, the term “analogs” refers to two nucleic acids that have the same or similar function, but that have evolved separately in unrelated organisms. As used herein, the term “orthologs” refers to two nucleic acids from different species, but that have evolved from a common ancestral gene by specification. Normally, orthologs encode polypeptides having the same or similar functions. As also used herein, the term “paralogs” refers to two nucleic acids that are related by duplication within a genome. Paralogs usually have different functions, but these functions may be related (Tatusov, Science 278 (1997), 631-637). Analogs, orthologs and paralogs of naturally occurring flagellins can differ from the naturally occurring flagellins, by post-translational modifications, by amino acid sequence differences, or by both. Post-translational modifications include in vitro chemical derivatization of polypeptides, e.g., acetylation, carboxylation, phosphorylation, or glycosylation, and such modifications may occur during polypeptide synthesis or processing or following treatment with isolated modifying enzymes. In particular, orthologs of the invention will generally exhibit at least 80-85%, more preferably, 85-90% or 90-95%, and most preferably 95%, 96%, 97%, 98% or even 99% identity or sequence identity with all or part of a naturally occurring flagellin sequence and will exhibit a function similar to a flagellin.

Alternatively, such polynucleotides can be prepared by genetic engineering or chemical synthesis.

Hybridizing polynucleotides may be identified and isolated by using the polynucleotides described herein/above or parts or reverse complements thereof, for instance by hybridization according to standard methods (see for instance Sambrook and Russell (2001), Molecular Cloning: A Laboratory Manual, CSH Press, Cold Spring Harbor, N.Y., USA). Polynucleotides comprising the same or substantially the same nucleotide sequence as indicated in SEQ ID NOs: 1, 3, 5, 7, 9, 11, 13, 15, 17, 19, 21, 23, 25, 27, 29, 31, 33, 35, 37, 39, 41 or 43 can, for instance, be used as hybridization probes. The fragments used as hybridization probes can also be synthetic fragments which are prepared by usual synthesis techniques, and the sequence of which is substantially identical with that of a polynucleotide according to the invention.

The molecules hybridizing with the polynucleotides of the invention also comprise fragments, derivatives and allelic variants of the above-described polynucleotides encoding a flagellin. Herein, fragments are understood to mean parts of the polynucleotides which are long enough to encode the described polypeptide, preferably showing the biological activity of a polypeptide of the invention as described above. In this context, the term derivative means that the sequences of these molecules differ from the sequences of the above-described polynucleotides in one or more positions, preferably within the preferred ranges of homology mentioned above.

Preferably, the degree of homology is determined by comparing the respective sequence with the nucleotide sequence of the coding region of SEQ ID NOs: 1, 3, 5, 7, 9, 11, 13, 15, 17, 19, 21, 23, 25, 27, 29, 31, 33, 35, 37, 39, 41 or 43. When the sequences which are compared do not have the same length, the degree of homology preferably refers to the percentage of nucleotide residues in the shorter sequence which are identical to nucleotide residues in the longer sequence. The degree of homology can be determined conventionally using known computer programs such as the DNASTAR program with the ClustalW analysis. This program can be obtained from DNASTAR, Inc., 1228 South Park Street, Madison, Wis. 53715 or from DNASTAR, Ltd., Abacus House, West Ealing, London W13 OAS UK (support@dnastar.com) and is accessible at the server of the EMBL outstation.

When using the Clustal analysis method to determine whether a particular sequence is, for instance, 80% identical to a reference sequence the settings are preferably as follows: Matrix: blosum 30; Open gap penalty: 10.0; Extend gap penalty: 0.05; Delay divergent: 40; Gap separation distance: 8 for comparisons of amino acid sequences. For nucleotide sequence comparisons, the Extend gap penalty is preferably set to 5.0.

Preferably, the degree of homology of the hybridizing polynucleotide is calculated over the complete length of its coding sequence which is described herein. It is furthermore preferred that such a hybridizing polynucleotide, and in particular the coding sequence comprised therein, has a length of at least 300 nucleotides, preferably at least 500 nucleotides, more preferably of at least 750 nucleotides, even more preferably of at least 1000 nucleotides and particularly preferred of at least 1500 nucleotides.

Preferably, sequences hybridizing to a polynucleotide according to the invention comprise a region of homology of at least 90%, preferably of at least 93%, more preferably of at least 95%, still more preferably of at least 98% and particularly preferred of at least 99% identity to an above-described polynucleotide, wherein this region of homology has a length of at least 500 nucleotides, more preferably of at least 750 nucleotides, even more preferably of at least 1000 nucleotides and particularly preferred of at least 1500 nucleotides.

Homology, moreover, means that there is a functional and/or structural equivalence between the corresponding polynucleotides or polypeptides encoded thereby. Polynucleotides which are homologous to the above-described molecules and represent derivatives of these molecules are normally variations of these molecules which represent modifications having the same biological function. They may be either naturally occurring variations, or mutations, and said mutations may have formed naturally or may have been produced by deliberate mutagenesis. Furthermore, the variations may be synthetically produced sequences. The allelic variants may be naturally occurring variants or synthetically produced variants or variants produced by recombinant DNA techniques. Deviations from the above-described polynucleotides may have been produced, e.g., by deletion, substitution, insertion and/or recombination.

The polypeptides encoded by the different variants of the polynucleotides of the invention possess certain characteristics they have in common. These include for instance biological activity, molecular weight, immunological reactivity, conformation, etc., and physical properties, such as for instance the migration behavior in gel electrophoreses, chromatographic behavior, sedimentation coefficients, solubility, spectroscopic properties, stability, pH optimum, temperature optimum etc.

The biological activity of a polypeptide of the invention, in particular the capacity to act as flagellin, can be tested as is known in the art.

The invention also relates to oligonucleotides specifically hybridizing to a polynucleotide of the invention. Such oligonucleotides have a length of preferably at least 10, in particular at least 15, and particularly preferably of at least 50 nucleotides. Advantageously, their length does not exceed a length of 1000, preferably 500, more preferably 200, still more preferably 100 and most preferably 50 nucleotides. The oligonucleotides of the invention can be used for instance as primers for amplification techniques such as the PCR reaction or as a hybridization probe to isolate related genes. The hybridization conditions and homology values described above in connection with the polynucleotide encoding a flagellin may likewise apply in connection with the oligonucleotides mentioned herein.

The polynucleotides of the invention can be DNA molecules, in particular genomic DNA or cDNA. Moreover, the polynucleotides of the invention may be RNA molecules. The polynucleotides of the invention can be obtained for instance from natural sources or may be produced synthetically or by recombinant techniques, such as PCR.

In another aspect, the present invention relates to recombinant nucleic acid molecules comprising the polynucleotide of the invention described above. The term “recombinant nucleic acid molecule” refers to a nucleic acid molecule which contains in addition to a polynucleotide of the invention as described above at least one further heterologous coding or non-coding nucleotide sequence. The term “heterologous” means that said polynucleotide originates from a different species or from the same species, however, from another location in the genome than said added nucleotide sequence. The term “recombinant” implies that nucleotide sequences are combined into one nucleic acid molecule by the aid of human intervention. The recombinant nucleic acid molecule of the invention can be used alone or as part of a vector.

In a preferred embodiment, the recombinant nucleic acid molecules further comprise expression control sequences operably linked to the polynucleotide comprised by the recombinant nucleic acid molecule, more preferably these recombinant nucleic acid molecules are expression cassettes. The term “operatively linked”, as used in this context, refers to a linkage between one or more expression control sequences and the coding region in the polynucleotide to be expressed in such a way that expression is achieved under conditions compatible with the expression control sequence.

Expression comprises transcription of the heterologous DNA sequence, preferably into a translatable mRNA. Regulatory elements ensuring expression in prokaryotic as well as in eukaryotic cells are well known to those skilled in the art. They encompass promoters, enhancers, termination signals, targeting signals and the like. Examples are given further below in connection with explanations concerning vectors. In the case of eukaryotic cells, expression control sequences may comprise poly-A signals ensuring termination of transcription and stabilization of the transcript; additional regulatory elements may include transcriptional as well as translational enhancers. It can be stated, that information processing (=transcription and translation) in archaea resembles much more the bacterial than the eukaryotic systems, which indicates that genetic manipulations can be done in archaea. Although, so far only some genetic markers (e.g. phenotypic markers, reporter genes) from archaea are known, it is believed that flagellins can be expressed in archaea. Accordingly, expression of a flagellin in its natural host under its endogenous promoter is envisaged. Expression can also be achieved by using a preferably strong constitutive or strong inducible promoter which is different from the promoter that normally controls expression of the flagellin of interest. Alternatively, expression in a heterologous archaeal host is believed to be feasible. The term “heterologous archaeal host” means that said archaeal host is different from the archaebacterium which expresses the flagellin of interest.

Moreover, vectors encoding the flagellins may be used to express said proteins. These vectors are, inter alia, in particular plasmids, cosmids, viruses, Yacs, Bacs, bacteriophages and other vectors commonly used in genetic engineering, which contain the above-described polynucleotides of the invention. In a preferred embodiment of the invention, the vectors of the invention are suitable for the transformation of fungal cells, cells of microorganisms such as yeast or bacterial cells, animal cells or of plant cells.

The vectors may further comprise expression control sequences operably linked to said polynucleotides contained in the vectors. These expression control sequence may be suited to ensure transcription and synthesis of a translatable RNA in prokaryotic or eukaryotic cells.

The expression of the polynucleotides of the invention in prokaryotic or eukaryotic cells, for instance in Escherichia coli, is interesting because it permits a more precise characterization of the biological activities of the encoded polypeptide. Moreover, it is possible to express these polypeptides in such prokaryotic or eukaryotic cells which are free from interfering polypeptides. In addition, it is possible to insert different mutations into the polynucleotides by methods usual in molecular biology (see for instance Sambrook and Russell (2001), Molecular Cloning: A Laboratory Manual, CSH Press, Cold Spring Harbor, N.Y., USA), leading to the synthesis of polypeptides possibly having modified biological properties. In this regard it is on the one hand possible to produce deletion mutants in which polynucleotides are produced by progressive deletions from the 5′ or 3′ end of the coding DNA sequence, and said polynucleotides lead to the synthesis of correspondingly shortened polypeptides as described herein.

On the other hand, the introduction of point mutations is also conceivable at positions at which a modification of the amino acid sequence for instance influences the biological activity or the regulation of the polypeptide.

For genetic engineering in prokaryotic cells, the polynucleotides of the invention or parts of these molecules can be introduced into plasmids which permit mutagenesis or sequence modification by recombination of DNA sequences. Standard methods (see Sambrook and Russell (2001), Molecular Cloning: A Laboratory Manual, CSH Press, Cold Spring Harbor, N.Y., USA) allow base exchanges to be performed or natural or synthetic sequences to be added. DNA fragments can be connected to each other by applying adapters and linkers to the fragments. Moreover, engineering measures which provide suitable restriction sites or remove surplus DNA or restriction sites can be used. In those cases, in which insertions, deletions or substitutions are possible, in vitro mutagenesis, “primer repair”, restriction or ligation can be used. In general, a sequence analysis, restriction analysis and other methods of biochemistry and molecular biology are carried out as analysis methods.

Additionally, the present invention also describes a method for producing genetically engineered host cells comprising introducing the herein above-described polynucleotides, recombinant nucleic acid molecules or vectors encoding archaeal flagellins into a host cell.

Thus, the present invention relates to a method for the production of a polypeptide encoded by the polynucleotide as described herein comprising culturing a host cell comprising said polynucleotide and recovering said polypeptide.

A further aspect of the invention is a polypeptide obtainable by the afore described method for the production of a polypeptide of the invention. Said polypeptide may be further modified. Modifications include glycosylation, acetylation, acylation, phosphorylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formulation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination; see, for instance, PROTEINS—STRUCTURE AND MOLECULAR PROPERTIES, 2nd Ed., T. E. Creighton, W. H. Freeman and Company, New York (1993); POST-TRANSLATIONAL COVALENT MODIFICATION OF PROTEINS, B. C. Johnson, Ed., Academic Press, New York (1983), pgs. 1-12; Seifter, Meth. Enzymol. 182 (1990); 626-646, Rattan, Ann. NY Acad. Sci. 663 (1992); 48-62.

The host can be transformed or transfected with a vector comprising the polynucleotide of the invention. Said host may be produced by introducing said vector or polynucleotide into a host cell which upon its presence in the cell mediates the expression of a protein encoded by the polynucleotide of the invention, wherein the nucleotide sequence and/or the encoded polypeptide is foreign to the host cell. Suitable host cells and vectors are described herein.

By “foreign” it is meant that the polynucleotide and/or the encoded polypeptide is either heterologous with respect to the host, which means that it is derived from a cell or organism with a different genomic background, or it is homologous with respect to the host but located in a different genomic environment than the naturally occurring counterpart of said polynucleotide. This means that, if the nucleotide sequence is homologous with respect to the host, it is not located in its natural location in the genome of said host, in particular it is surrounded by different genes. In this case the polynucleotide may be either under the control of its own promoter or under the control of a heterologous promoter. The location of the introduced polynucleotide or the vector can be determined by the skilled person by using methods well-known to the person skilled in the art, e.g., Southern Blotting. The vector or polynucleotide according to the invention which is present in the host may either be integrated into the genome of the host or it may be maintained in some form extrachromosomally. In this respect, it is also to be understood that the polynucleotide of the invention can be used to restore or create a mutant gene via homologous recombination.

The useful flagellin may be produced in host cells, in particular prokaryotic or eukaryotic cells, genetically engineered with the above-described polynucleotides, recombinant nucleic acid molecules or vectors of the invention or obtainable by the above-mentioned method for producing genetically engineered host cells, and to cells derived from such transformed cells and containing a polynucleotide, recombinant nucleic acid molecule or vector of the invention. In a preferred embodiment the host cell is genetically modified in such a way that it contains a polynucleotide stably integrated into the genome. Preferentially, the host cell of the invention is a bacterial, yeast, fungus, plant or animal cell.

More preferably the polynucleotide can be expressed so as to lead to the production of a flagellin polypeptide. An overview of different expression systems is for instance contained in Methods in Enzymology 153 (1987), 385-516, in Bitter (Methods in Enzymology 153 (1987), 516-544) and in Sawers (Applied Microbiology and Biotechnology 46 (1996), 1-9), Billman-Jacobe (Current Opinion in Biotechnology 7 (1996), 500-4), Hocknev (Trends in Biotechnology 12 (1994), 456463), Griffiths (Methods in Molecular Biology 75 (1997), 427-440). An overview of yeast expression systems is for instance given by Hensing (Antonie van Leuwenhoek 67 (1995), 261-279), Bussineau (Developments in Biological Standardization 83 (1994), 13-19), Gellissen (Antonie van Leuwenhoek 62 (1992), 79-93), Fleer (Current Opinion in Biotechnology 3 (1992), 486-496), Vedvick (Current Opinion in Biotechnology 2 (1991), 742-745) and Buckholz (Bio/Technology 9 (1991), 1067-1072). Expression vectors have been widely described in the literature. As a rule, they contain not only a selection marker gene and a replication-origin ensuring replication in the host selected, but also a bacterial or viral promoter, and in most cases a termination signal for transcription. Between the promoter and the termination signal there is in general at least one restriction site or a polylinker which enables the insertion of a coding DNA sequence. The DNA sequence naturally controlling the transcription of the corresponding gene can be used as the promoter sequence, if it is active in the selected host organism. However, this sequence can also be exchanged for other promoter sequences. It is possible to use promoters ensuring constitutive expression of the gene and inducible promoters which permit a deliberate control of the expression of the gene. Bacterial and viral promoter sequences possessing these properties are described in detail in the literature. Regulatory sequences for the expression in microorganisms (for instance E. coli, S. cerevisiae) are sufficiently described in the literature. Promoters permitting a particularly high expression of a downstream sequence are for instance the T7 promoter (Studier et al., Methods in Enzymology 185 (1990), 60-89), lacUV5, trp, trp-lacUV5 (DeBoer et al., in Rodriguez and Chamberlin (Eds), Promoters, Structure and Function; Praeger, New York, (1982), 462-481; DeBoer et al., Proc. Natl. Acad. Sci. USA (1983), 21-25), Ip1, rac (Boros et al., Gene 42 (1986), 97-100). Inducible promoters are preferably used for the synthesis of polypeptides. These promoters often lead to higher polypeptide yields than do constitutive promoters. In order to obtain an optimum amount of polypeptide, a two-stage process is often used. First, the host cells are cultured under optimum conditions up to a relatively high cell density. In the second step, transcription is induced depending on the type of promoter used. In this regard, a tac promoter is particularly suitable which can be induced by lactose or IPTG (=isopropyl-β-D-thiogalactopyranoside) (DeBoer et al., Proc. Natl. Acad. Sci. USA 80 (1983), 21-25). Termination signals for transcription are also described in the literature.

The transformation of the host cell with a polynucleotide or vector according to the invention can be carried out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990. The host cell is cultured in nutrient media meeting the requirements of the particular host cell used, in particular in respect of the pH value, temperature, salt concentration, aeration, antibiotics, vitamins, trace elements etc. The polypeptide according to the present invention can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Polypeptide refolding steps can be used, as necessary, in completing configuration of the polypeptide. Finally, high performance liquid chromatography (HPLC) can be employed for final purification steps.

As documented in the appended examples and figures, the flagellins useful in context of the present invention comprise a “consensus sequence”. Accordingly, the flagellin to be employed in this invention comprises in its amino acid sequence the consensus sequence AxGIGTLIVFIAMVLVAAVAA.

The person skilled in the art is readily in the position to obtain a flagellin/flagellin protein preparation. A corresponding method may comprise the following steps

(a) culturing archaea cells with flagella;
(b) shearing the flagella from said cells;
(c) purifying said flagella;
(d) isolating the flagellin from said flagella.

In general, culturing of archaea can be done by applying methods known in the art which may be somewhat adjusted by the skilled person to the respective archaebacterium, if deemed to be necessary. Shearing of flagella follows procedures known in the art which are exemplified in the appended Examples. Purifying of flagella can be done, for example, as described in the appended Examples. Isolating flagellin from flagella can be, for example, done by using denaturing agents such as SDS, for example, 0.1% SDS, Triton, for example Triton X-100 and/or purification via e.g. size exclusion chromatography. Further purification techniques that may be used are, for example, anion or cation exchange chromatography, phosphocellulose chromatography, hydrophobic interaction chromatography, affinity chromatography, hydroxylapatite chromatography and the like. Polypeptide refolding steps can be used, as necessary, in completing configuration of the polypeptide. Finally, high performance liquid chromatography (HPLC) can be employed for final purification steps.

Flagellar proteins can be purified from isolated flagella by the following general procedure:

Flagella are denatured by various treatments into the flagellin monomers which can be purified in solutions containing those denaturing agents via e.g. chromatographic procedures, especially those separating the monomers according to their size (especially HPLC purification has to be noted here). For denaturation of flagella into monomers a 60 min treatment at 25° C. with a final concentration of the following detergents can be used: 0.1% SDS; or 0.05% Triton X100; or 0.05% CTAB. Solubilization of flagella into monomers also can be achieved by a 60 min treatment at 80° C. with a final concentration of 1.5 M guanidine hydrochloride.

As documented in the appended examples, a partial preferred flagellin to be employed as adhesive material is obtainable from Pyrococcus furiosus (P. furiosus), more preferably from P. furiosus is P. furiosus Vc1, and particularly preferred from P. furiosus Vc1 as previously deposited under DSM3638 with the “Deutsche Sammiung von Mikroorganismen und Zellkulturen (DSMZ)”, Mascheroder Weg 1b, 38124 Braunschweig, Germany. The corresponding strain was also described in Fiala (1986), Int. J. Syst. Bacteriol. 36, 573.

The flagellin to be employed in context of this invention is preferably a flagellin protein of 30 kDa protein, as deduced by SDS-PAGE analysis on a 12.5% gel. Corresponding methods are provided in the experimental part.

The particular preferred flagellin is encoded by a nucleotide sequence as shown in SEQ ID NO: 1 or comprises an amino acid sequence as shown in SEQ ID NO: 2.

In a further aspect, the present application relates to a composition comprising the adhesive material as described herein or comprising at least one protein obtained or obtainable from flagella from archaea as described herein.

The term “composition”, as used in accordance with the present invention, relates to compositions which comprise at least one adhesive material or at least one protein obtained or obtainable from flagella from archaea. It may, optionally, comprise further ingredients useful as adhesive material. The composition may be in solid or liquid form and may be, inter alia, in the form of (a) powder(s), (a) solution(s) or the like. In a preferred aspect, the composition described herein is a pharmaceutical composition which may in particular be useful for the medical applications/devices as mentioned herein.

In a still further aspect, the present invention relates to antibodies which specifically bind to the proteins, variants or derivatives or fragments thereof as described herein. The antibody of the present invention can be, for example, polyclonal or monoclonal. The term “antibody” also comprises derivatives or fragments thereof which still retain the binding specificity such as a Fab, F(ab′)₂, Fv or scFv fragment. Techniques for the production of antibodies are well known in the art and described, e.g. in Harlow and Lane “Antibodies, A Laboratory Manual”, CSH Press, Cold Spring Harbor, 1988. The present invention furthermore includes chimeric, single chain and humanized antibodies, as well as antibody fragments as mentioned above; see also, for example, Harlow and Lane, loc. cit. Various procedures are known in the art and may be used for the production of such antibodies and/or fragments. Thus, the (antibody) derivatives can be produced by peptidomimetics. Further, techniques described for the production of single chain antibodies (see, inter alia, U.S. Pat. No. 4,946,778) can be adapted to produce single chain antibodies to polypeptide(s) of this invention. Also, transgenic animals may be used to express humanized antibodies to polypeptides of this invention. For the preparation of monoclonal antibodies, any technique which provides antibodies produced by continuous cell line cultures can be used. Examples for such techniques include the hybridoma technique (Köhler and Milstein Nature 256 (1975), 495-497), the trioma technique, the human B-cell hybridoma technique (Kozbor, Immunology Today 4 (1983), 72) and the EBV-hybridoma technique to produce human monoclonal antibodies (Cole et al., Monoclonal Antibodies and Cancer Therapy, Alan R. Liss, Inc. (1985), 77-96). Techniques describing the production of single chain antibodies (e.g., U.S. Pat. No. 4,946,778) can be adapted to produce single chain antibodies to immunogenic polypeptides as described above. Accordingly, in context of the present invention, the term “antibody molecule” relates to full immunoglobulin molecules as well as to parts of such immunoglobulin molecules. Furthermore, the term relates, as discussed above, to modified and/or altered antibody molecules, like chimeric and humanized antibodies. The term also relates to monoclonal or polyclonal antibodies as well as to recombinantly or synthetically generated/synthesized antibodies. The term also relates to intact antibodies as well as to antibody fragments thereof, like, separated light and heavy chains, Fab, Fab/c, Fv, Fab′, F(ab′)2. The term “antibody molecule” also comprises bifunctional antibodies and antibody constructs, like single chain Fvs (scFv) or antibody-fusion proteins. It is also envisaged in context of this invention that the term “antibody” comprises antibody constructs which may be expressed in cells, e.g. antibody constructs which may be transfected and/or transduced via, inter alia, viruses or vectors. Of course, the antibody of the present invention can be coupled, linked or conjugated to detectable substances.

As discussed above several uses of the flagellin provided herein are now envisaged since it was surprisingly found that also in vivo the “flagella” of P. furiosus are not only made for motion (swimming) but are also used to adhere to different surfaces. Accordingly, the present invention provides for archaea flagellin(s) as adhesive material/glue as described herein.

The Figures show:

FIG. 1

A model for localization of the different Fla proteins in flagella of Methanococcus vannielii proposed by Bardy (2003; Microbiology 149:295-304).

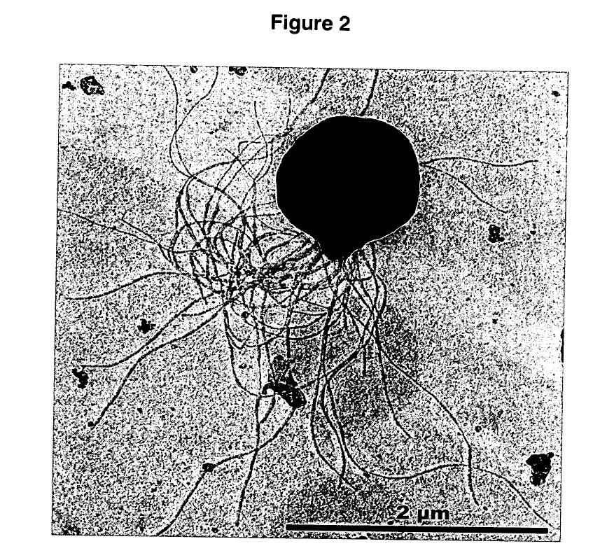

FIG. 2

TEM picture of a cell of Pyrococcus furiosus Vc1. Multiple surface appendages named flagella are visible on the cell.

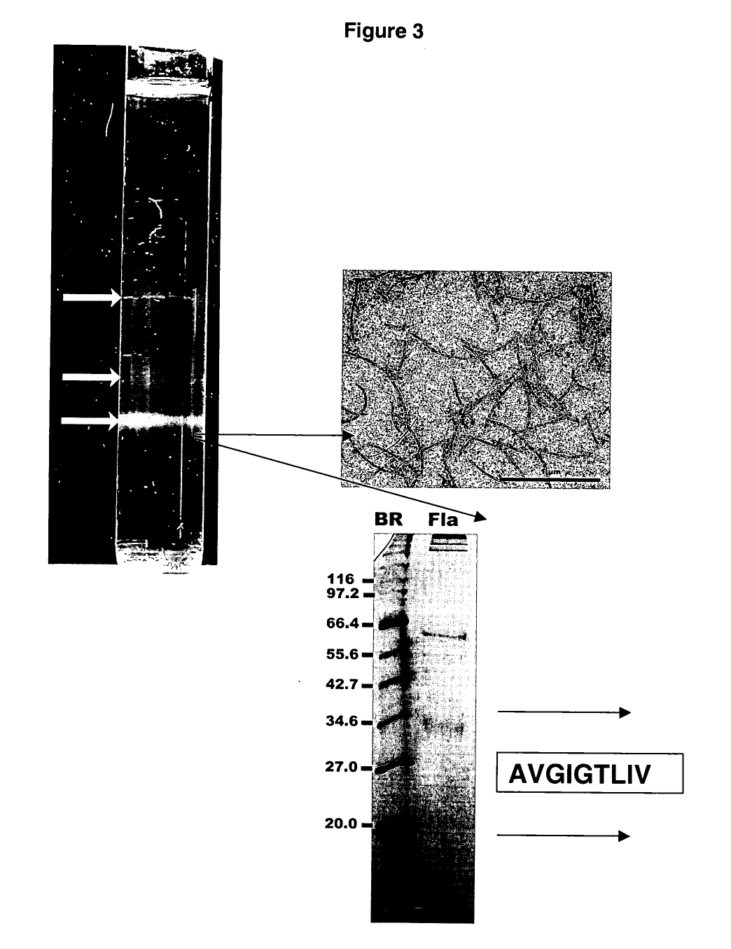

FIG. 3

Biochemical analysis of a flagella preparation obtained via shearing from cells and purification by isopycnic cesium chloride centrifugation.

The lowest band of the cesium chloride gradient contained pure flagella as was shown by TEM analysis. This preparation resulted in two main proteins as shown by SDS-PAGE analysis (lane Fla). Both bands migrating at 30 kDa and 60 kDa (size standards are given in lane BR) were sequenced by Edman degradation to result in the same N-terminus of AVGIGTLIV.

FIG. 4

Comparison of the N-terminal regions of various Fla proteins from archaea flagella according to Thomas (2001; FEMS Microbiol. Rev. 25:147-174). Indicated is the signal peptide, the signal peptidase cleavage site and the N-terminal 20 amino acids of the mature proteins. From those data a consensus N-terminal sequence was deduced. It is obvious that FlaB from Pyrococcus furiosus does possess exactly this N-terminal consensus sequence. Amino acids underlined in the P. furiosus FlaB protein have been determined via Edman degradation of the purified monomer from isolated flagella (see FIG. 3).

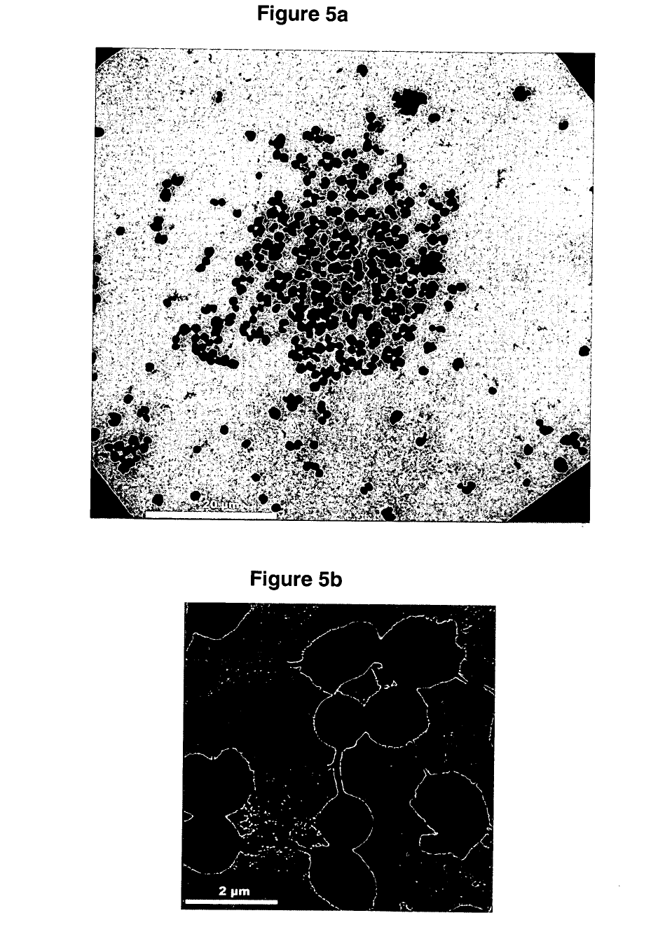

FIG. 5

Growth of Pyrococcus furiosus on gold grids used for TEM. Gold grids used as support for TEM studies were added (in a Teflon holder) to serum bottles used for growing P. furiosus. Those grids were contrasted with uranyl acetate and could be analysed via phase contrast light microscopy or via transmission electron microscopy.

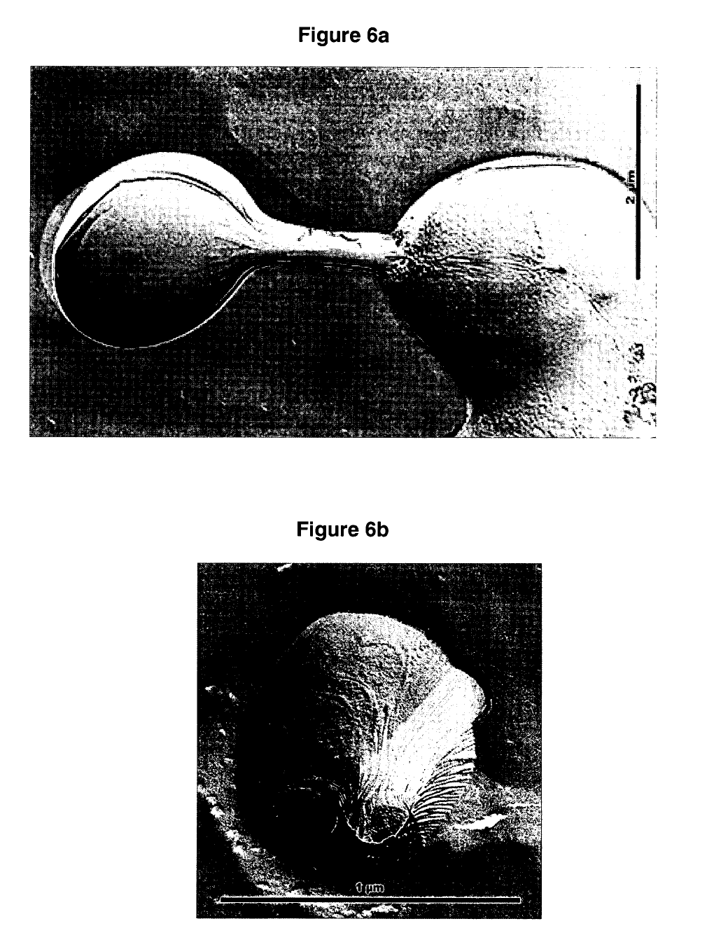

FIG. 6

Analysis of cell-cell connections using freeze etch technique. Cells grown in liquid medium were applied on a gold holder and frozen in liquid nitrogen. The sample was broken whilst being frozen and parts of it exposed to the surface via sublimation of surface water. Thereafter the sample was sputtered with platinum and carbon. Organic cell remains were solubilized with sulphuric acid from the platinum/carbon replica and transferred to a new TEM grid. It is evident from FIG. 6a that flagella assemble into a cable-like structure connecting two P. furiosus cells (in very a few instances not only pairs, but also triplets of cells were observed). In FIG. 6b a cross-section of such a cable clearly demonstrates that very many flagella aggregate to form one cable.

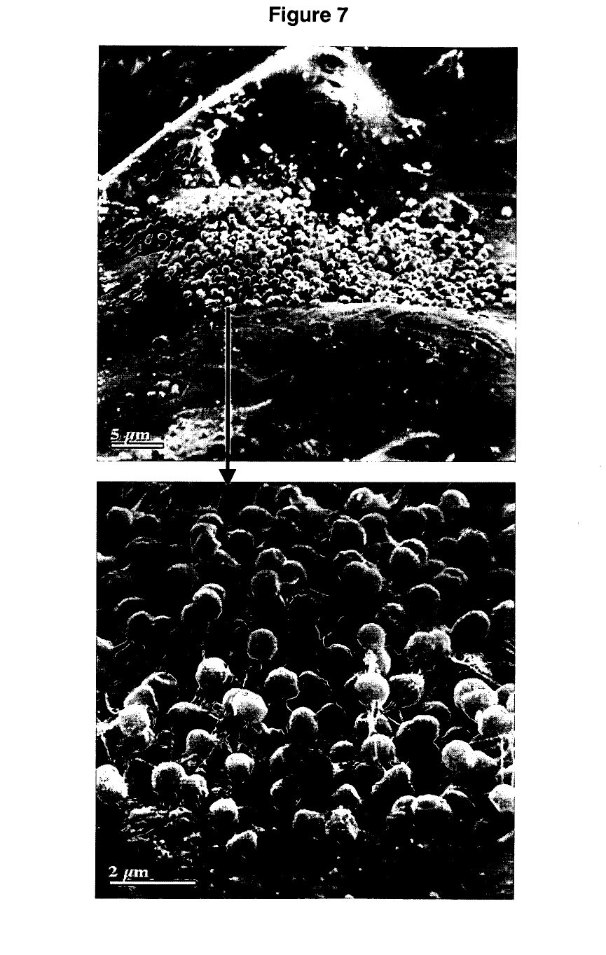

FIG. 7

Microcolony growing on a sand grain collected at the biotope from which Pyrococcus furiosus Vc1 originally was isolated. Sterilized sand grains were added to serum bottles; after addition and sterilization of medium P. furiosus was inoculated into those serum bottles. The sand grains were processed for SEM (glutardialdehyde fixation; dehydration in a graded series of acetone solutions; critical point drying with CO₂; mounting on copper foils; sputtering with platinum) and analysed in a Hitachi model S-4100 field emission scanning electron microscope.

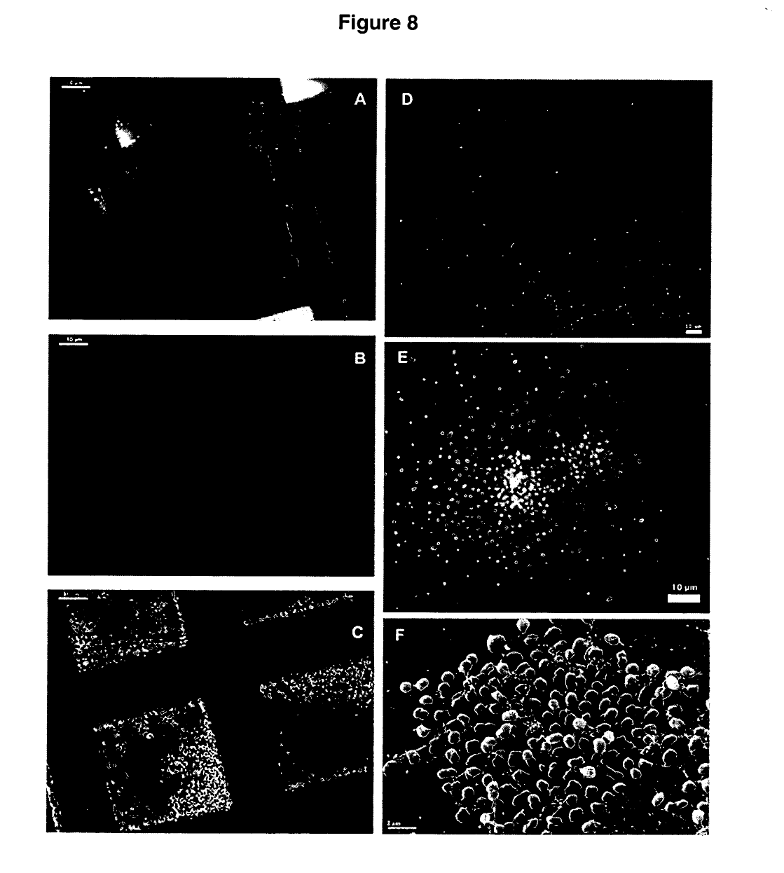

FIG. 8

Microscopic demonstration of adherence of Pyrococcus furiosus Vc1 to various surfaces and proof of binding specificity for flagella. Binding assays were performed as outlined above (e.g. FIGS. 5 and 7); detection of bound cells was either by DAPI staining (FIG. 8A; FIG. 8B; FIG. 8D; FIG. 8E), or by phase contrast microscopy (FIG. 8C). FIG. 8F shows a microcolony of P. furiosus Vc1 as detected by SEM. FIG. 8A demonstrates that P. furiosus Vc1 adheres to gold grids. FIG. 8B shows that this binding is specifically mediated by flagella, since addition of antibodies against flagella did remove bound cells. The specificity of the antiserum used is demonstrated in FIG. 8C: addition of antibodies against an intracellular protein of P. furiosus Vc1 (RNA polymerase) does not remove bound cells. As. FIG. 8D shows P. furiosus Vc1 does adhere very well to polycarbonate in an evenly distributed manner. Growth on Si-wafers, on the other hand is more in form of microcolonies as shown in FIG. 8E and FIG. 8F. Size bars are 10 μm in FIGS. 8A to 8E and 2 μm in FIG. 8H.

The invention is illustrated by but not limited to the following examples.

### EXAMPLE 1

### Materials and Methods Used in this Study

### Growth of Cells and Preparation of Flagella

Pyrococcus furiosus Vc1 (DSM 3638 as deposited 1986 with the DSMZ, Braunschweig, Germany) was cultured anaerobically in Stetters modified “SME-medium” (1983; System. Appl. Microbiol. 4:535-551) at 90° C. in serum bottles. Cell masses were grown anaerobically at 95° C. in a 50-liter fermentor (Bioengeneering, Wald, Switzerland) pressurized with 100 kPa of N₂/CO₂ (80:20) to early stationary phase. The cell suspension was centrifuged for 30 min (3,000 g, 4° C., Sorvall Centrifuge). The pellet was sheared with an Ultraturrax T25 (IKA-Werke, Staufen, Germany) for 1 min with 13,000 rpm and 10 s with 20,500 rpm and afterwards centrifuged twice for 15 min (16,000 g, 4° C., Sorvall Centrifuge). The supernatant containing the flagella was resuspended by diluting in a small volume of 0.1 M HEPES-buffer (pH 7). Further purification on a CsCl-gradient (0.45 g/ml) by centrifugation for 48 h (SW60-Ti rotor at 48,000 rpm, 4° C., Beckman Optima LE-80K ultracentrifuge) resulted in three fractions, that were isolated and dialysed exhaustivly against 5 mM HEPES-dialysis-buffer (pH 7) at 4° C. The isolated fractions were analysed by TEM and that containing the purified flagella was used for further tests.

### Biochemical Characterization of Flagella

Protein samples were resolved by electrophoresis on a 12.5% sodium dodecyl sulfate-polyacrylamide gel (SDS-PAGE—Lämmli (1970; Nature 227:680-685) and the proteins were stained with Coomassie Brilliant Blue 250 and destained with 30% methanol/10% acetic acid, or as described by Blum (1987; Electrophoresis 8:93-99) via silver staining. The method used for detection of protein glycosylation has been described by Zacharius (1969; Anal. Biochem. 30:148-152). N-terminal sequencing was performed by the central protein analytic facility of the Biology Department of the University of Regensburg.

### Adherence Studies—Growth on Gold Grids for Transmission Electron Microscopy (TEM)

Rieger (1998; Dissertation at the University of Regensburg; Title: Elektronemikroskopische und biochemische Untersuchungen zum Aufbau des Netzwerks von Pyrodictium) has developed methods in our labs to study growth of microorganisms directly on gold grids. In principle gold grids are placed in small Teflon holders into serum bottles containing anaerobic medium for growing e.g. hyperthermophilic archaea. For transmission electron microscopy cells were fixed with 2.5% glutardialdehyde (final concentration) for 30 min at room temperature. In the case of cell- or flagella-suspensions a drop was placed for 30 s on a 200-mesh cooper grid (Piano, Wetzlar, Germany), which was covered by a carbon film. Cell suspensions were either shadowed with a Pt/C gun at 15° (DFE 50, Cressington Ltd., Watford, UK) or negatively stained for 1 min with 2% uranyl acetate. Flagella in almost all cases were shadowed with a Pt/C gun at 150 (DFE 50, Cressington Ltd., Wafford, UK). Micrographs were taken with a Philips CM 12 TEM (Philips, Eindhoven, Netherlands) operating at 200 kV and a slow-scan CCD-camera (TEM 1000, TVIPS-Tietz, Gauting) with 200 ms exposure time.

### Characterization of Cell-Cell Connections Using Freeze Etching Techniques and TEM

Rachel (2002; Archaea 1:9-18) has described the methods used for freeze-etching experiments. In principal cells from supernatants were harvested by centrifugation, loaded onto a gold holder and plunged into liquid nitrogen. The samples were cut with a cold knife (T<−185° C.) in a CFE-50 freeze-etch-unit (Cressington, Wafford; U.K.), then shadowed (1 nm Pt/C 45°, 10 nm C 90°) and cleaned on 70% H₂SO₄. Micrographs were taken with a Philips CM 12 TEM (Philips, Eindhoven, Netherlands) operating at 200 kV and a slow-scan CCD-camera (TEM 1000, TVIPS-Tietz, Gauting) with 200 ms exposure time.

Adherence Studies—Growth on Sand Grains from the Original Habitat Using Scanning Electron Microscopy (SEM)

For these studies sand grains from the beach of Porto di Levante, Italy were added to serum bottles instead of the gold grids used for TEM studies. After incubation, the solids with adhering cells were collected and stored in SME-Medium with 2.5% glutardialdehyde (final concentration). After washing in double-distilled water and dehydration with a graded series of acetone solutions, cells were critical point dried with liquid CO₂, mounted with conductive tabs (Plano, Wetzlar, Germany) and sputter coated with platinum by using a magnetron sputter coater (model SCD 050; BAL-TEC, Walluf, Germany), which produces a layer about 5 to 7 nm thick. All pictures were obtained with energy-dispersive X-ray microanalysis.

### EXAMPLE 2

### Structural Analysis of P. furiosus Flagella

P. furiosus Cells Possess Multiple Flagella

As FIG. 2 shows many flagella can be observed on the surface of P. furiosus cells, with a length of 2 to 3 μm, some are even 7 μm long. Systematic investigations indicated that their number is highest at late exponential to stationary phase.

P. furiosus Flagella are Composed of Only One Flagellin

After shearing flagella from cells and purification via isopycnic cesium chloride centrifugation we obtained a preparation consisting of filaments ca. 1 μm in length with a diameter of 9-10 nm. These filaments are composed to >95% of one protein. In FIG. 3 such a preparation was analysed via SDS-PAGE (12.5% polyacrylamide) the two prominent protein bands at 30 and 60 kDa were analysed via protein sequencing; in both cases the same amino-terminal sequence of AVGIGTLIV was obtained. The 60 kDA protein therefore is a dimer of the 30 kDa monomer. Proteins migrating at very high molecular mass are multimers of the 30 kDa monomer, because their detection varied with different denaturation conditions.

The amino-terminal sequence obtained for the protein of which P. furiosus flagella are composed correlate completely with flagellins of other archaea. It has to be noted that in many cases this “identification” as flagellin is only via homologies to the proven flagellins of M. voltae. In FIG. 4 we show a comparison of the N-terminus of a few Fla sequences of different archaea with those of P. furiosus. It is evident that flagellins of archaea possess the signature sequence AXGIGTLIV at their amino terminus, identifying our protein as flagellin.

The 30 kDa protein did react specifically in a PAS (perjodat acid-Schiff) staining reaction, indicating that the flagellin of P. furiosus is a glycoprotein as has been reported for most archaeal flagellins. No biochemical data as for specific glycosylation sites are known for any archaeal flagellins, with the exception of H. salinarum which was analysed by Wieland (1985; J. Biol. Chem. 260:15180-15185) and M. voltae which was analysed by Voisin (2005; J. Biol. Chem. 280:16586-16593).

### EXAMPLE 3

### Functional Analysis of P. furiosus Flagella

Flagella of P. furiosus Enable the Cells to Adhere to Gold Grids Used for TEM

During our attempts to develop techniques to study the three-dimensional structure of P. furiosus flagella (via tomography) we realized that P. furiosus cells adhered to gold grids used for TEM. Light microscopic studies of such gold grids (which were incubated in serum bottles used for growth) indicated that cells grew in concentrations on the gold grids which were much higher than in the liquid supernatant. Higher resolutions using TEM, clearly indicated that the cells growing on gold grids did express flagella to a high degree. Two other observations were stunning: up to 5% of all cells showed some cell-cell connections; in some cases flagella seemed to aggregate into cable like structures.

The case of these cell-cell connections was studied using freeze etch techniques. The data obtained very clearly demonstrate that cell-cell connections are made from a multitude of flagella aggregating into a cable-like structure—see FIG. 6a. In a few cases we obtained preparations in which those cables were broken to allow a view on their cross axis; the single filaments in the cables had the diameter of flagella—see FIG. 6b.

Adherence of P. furiosus Cells to Sand Grains of its Natural Habitat

P. furiosus Vc1 originally was isolated from a marine sample taken at Porto di Levante (Italy) at a depth of a few meter. Since hot water emerging from the sea ground should move the cells off regions possessing the optimal growth temperature we speculated that the archaeum might adhere to sand grains of its natural habitat. By incubating some sand grains in serum bottles used for growing P. furiosus and preparing the grains via glutardialdehyde fixation for SEM it was shown that this is the case. P. furiosus did grow on the sand grains in a biofilm-like manner, allowing the development of microcolonies of the archaeum—see FIG. 7. The cells are connected by a multitude of cell surface appendages which in some cases are aggregated to a certain amount. The thinnest filaments had an estimated diameter of ca. 10 nm, which correlates very well with the value obtained for flagella using TEM. In addition the filaments were connected to the sand grains, not only at their tips but also over a distance of up to 1 μm. Some cells in such microcolonies again did show the cell-cell connections we had seen already before. Control experiments in which the sand grains were cleaned by sulphuric acid clearly showed that the sand grains had not to be “precoated” by some unknown substance to allow binding.

Adherence of P. furiosus to various surfaces

In these studies P. furiosus Vc1 was grown in serum bottles to which small pieces (ca. 15×10 mm) of different materials were added before addition of medium. After growth for 12 to 15 hours at 90° C. the serum bottles were opened, the solid surfaces removed by tweezers and stained for adhering cells using the dsDNA-specific fluorescence dye DAPI. Detection was by an Olympus BX50 fluorescence microscope; this approach allowed detection also for non-transparent materials since the UV-light used for detection is provided through the objective—see FIG. 8. The following materials were tested: gold-, nickel-, and copper-grids for TEM; stainless steel (V4A quality used for fermentors); household aluminium foil; plexiglass, polycarbonate, polyvinylchloride and nylon (various labware-consumables); enamel (coating of fermentors used for growth of P. furiosus); various types of glasses; mica (glass substitute for light-microscopy); Si-wafers (Infineon company). Strength of adherence was scored by numbers of cells detected per 15 mm² of surface; we did note different growth characteristics of P. furiosus cells for different surfaces—see Table 1. The final proof that flagella are responsible for binding of P. furiosus Vc1 to various surface came from experiments in which solids with adhering cells of P. furiosus were treated at room temperature with antibodies (diluted in sterile medium). After two washing steps with medium no adhering cells could be detected if antibodies raised against purified flagella (preparation seen in FIG. 3) were used, whereas antibodies raised against the intracellular P. furiosus RNA polymerase did not remove adherent cells.

TABLE 1
Adherence of Pyrococcus furiosus to various surfaces
Summary of adherence studies of Pyrococcus furiosus Vc1
to various surfaces. Binding assays were performed as outlined
in FIGS. 5, 7, and 8; detection was via DAPI staining. Results
were scored as: −, if only rarely single cells were observed;
(+), if less than 50 cells were observed per 15 mm² of
surface; +, if at least 100 cells were observed per 15 mm² of
surface; ++, if 100 to 1000 cells were observed per 15 mm²
of surface; +++, if dense growth with >> 1000 cells
per 15 mm² of surface were observed.

| | adherence | |
|---|---|---|
| Material | strength | growth pattern |
| Gold | +++ | very dense growth in microcolonies |
| Nickel | +++ | very dense growth in microcolonies |
| Copper | +++ | very dense growth, evenly |
| | | distributed |
| Steel | (+) | few cells |
| Aluminium | + | few cells in very small |
| | | microcolonies |
| Enamel | + | few cells |
| Glass | − | single cells |
| Si-wafer | ++ | dense growth in microcolonies |
| Mica | (+) | few cells at edge of single mica |
| | | layers |
| PVC | + | single cells in loose growth |
| Polycarbonate | +++ | very dense growth, evenly |
| | | distributed |
| Nylon | +++ | very dense growth, plus |
| | | microcolonies |
| Plexiglass | +++ | very dense growth in colonies |
| Wood | + | very small microcolonies |
| Quartz | +++ | very dense growth in microcolonies |

### EXAMPLE 4

### Temperature Experiment with Purified Flagella

Flagella were prepared (from cells growing at 95° C.) as described in example 1. They were incubated in 5 mM HEPES-buffer for various times (5 min; 10 min; 15 min; 30 min) at different temperatures (25° C.; 50° C.; 60° C.; 70° C.; 80° C.; 90° C.; 100° C.; 121° C.). Analysis was by TEM and SDS-PAGE. The electron microscopic data indicated no loss of function even after a 30 min treatment at 121° C., i.e. flagella were present still as long filaments. SDS-PAGE also did not indicate any loss of function because the samples had to be cooked in buffer containing 2.5% α-mercaptoethanol to dissociate flagella into the monomers.

Accordingly, the present invention provides for the first evidence that the cell surface organelles of archaea, in particular of P. furiosus are made not (only) for swimming but enable the archaeum to adhere to each other and to different surfaces, like gold grids, sand grains and various others as mentioned herein.

From this it is suggested that the Fla protein(s) of archaea, in particular of P. furiosus can be used as a molecular glue in various applications.

Flagellin sequences from archaea
(flagellin from flagella)

Pyrococcus furiosus FlaB

Nucleotide sequence
Atgaagaaaggagcaattggtatcggaacgctcatcgtcttcatcgcaat
ggtgcttgttgcggcagtagcagcaggtgtgctaatagcaacaagtggat
atttgcagcagaaggccatggccacaggtagacagacaacccaggaggtt
gcaagtggaatcaaggttactggtgtgttcggctatatcaatggcactcc
ccctggagcctcaaacataagcaggattgtcatatatgttgctccaaatg
cagggagtagtggaattgacttaagatatgtaaaaatagtgttaagcgat
gggaaaagaatggcagtgtacaggtattacgatccaaaggaggatggaag
ctcagacctaaagccagaatacattcactacaaaggagatatacctaaca
tatttgcttatggagagtgggaaccctactacaaaaacaagaagccacag
atatctggagaatacatcaccgataatattaacgtaagtgcagtttggtg
gaacctctacagtgcctacaacaactcaagcaagctactcttcgggattg
cggtagttcaagatggggacaacagccttagcgatccacaacatccaaca
ttaagctggggagacttagcagccctaatgatatggactttcccattcga
cgatgacaataatatctccaacggtttcgggctaagaccaggaacaaaga
ttataggaaaggtaattccagagagcggagctgctggtgttatcgacttc
acaactccctctacatatacccaaaacttaatggaacttcaatga
(SEQ ID NO: 1)
Amino acid sequence
MKKGAIGIGTLIVFIAMVLVAAVAAGVLIATSGYLQQKAMATGRQTTQEV
ASGIKVTGVFGYINGTPPGASNISRIVIYVAPNAGSSGIDLRYVKIVLSD
GKRMAVYRYYDPKEDGSSDLKPEYIHYKGDIPNIFAYGEWEPYYKNKKPQ
ISGEYITDNINVSAVWWNLYSAYNNSSKLLFGIAVVQDGDNSLSDPQHPT
LSWGDLAALMIWTFPFDDDNNISNGFGLRPGTKIIGKVIPESGAAGVIDF
TTPSTYTQNLMEL
(SEQ ID NO: 2)

Halobacterium sp. NRC1 FlaB1

Nucleotide sequence
Atgttcgagttcatcactgacgaagacgagcgcggccaagtggggatcgg
cacgctcatcgtgttcatcgcgatggtgctggtcgccgcgatcgccgccg
gcgtcctcatcaacaccgccggctacctccaatccaaggggtcggcaacc
ggtgaggaagcctccgcacaggtctccaaccgcatcaacatcgtctccgc
gtacggcaacgtcaacaacgagaaggtcgactacgtgaacctcaccgtgc
gccaggccgccggagccgacaacatcaacctcacgaaatccacgatccag
tggatcggcccggacagagccaccaccctgacgtactcgtcgaacagccc
gagttcgctgggtgaaaacttcaccaccgaatccatcaagggcagcagcg
ccgacgtgctggtcgaccagtccgaccgcatcaaggtcatcatgtacgcc
agcggcgtcagctccaacctcggcgctggtgacgaggtgcagctgacggt
gaccacgcagtacggctcgaaaaccacctactgggcgcaagtccctgaat
cgctcaaggacaaaaacgccgtcacactataa
(SEQ ID NO: 3)
Amino acid sequence
MFEFITDEDERGQVGIGTLIVFIAMVLVAAIAAGVLINTAGYLQSKGSAT
GEEASAQVSNRINIVSAYGNVNNEKVDYVNLTVRQAAGADNINLTKSTIQ
WIGPDRATTLTYSSNSPSSLGENETTESIKGSSADVLVDQSDRIKVIMYA
SGVSSNLGAGDEVQLTVTTQYGSKTTYWAQVPESLKDKNAVTL
(SEQ ID NO: 4)

Halobacterium sp. NRC1 FlaB2

Nucleotide sequence
Atggtgctggtcgccgcgatcgccgccggcgtcctcatcaacactgccgg
ctacctccaatccaaggggtccgcaactggtgaggaagcctccgcacagg
tctccaaccgcatcaacatcgtctccgcgtacggcaacgtggacacgtct
ggctcaaccgaggtagtcaattacgcgaacctgacggtgcgccaggccgc
tggggctgacaacatcaacctcagcaaatccacgatccagtggatcggcc
cggacaccgccactaccttgacctacgacgggactactgccgacgccgag
aacttcaccacgaattcgattaagggcgacaacgcggacgtgctggttga
tcagtccgaccgcatcgagatcgtcatggacgcggccgagatcaccacca
atggactgaaggctggcgaagaggtccagctgacagtgaccacgcagtac
ggctcgaaaaccacctactgggcgaacgttcctgagtcgctcaaggacaa
aaacgcagtcacgctataa
(SEQ ID NO: 5)
Amino acid sequence
MVLVAAIAAGVLINTAGYLQSKGSATGEEASAQVSNRINIVSAYGNVDTS
GSTEVVNYANLTVRQAAGADNINLSKSTIQWIGPDTATTLTYDGTTADAE
NFTTNSIKGDNADVLVDQSDRIEIVMDAAEITTNGLKAGEEVQLTVTTQY
GSKTTYWANVPESLKDKNAVTL
(SEQ ID NO: 6)

Methanococcus vannielii FlaB1

Nucleotide sequence
Atgagtgtaaaaaatttcatgaataacaagaaaggtgactctggaatcgg
caccttgattgttttcattgcaatggtattggttgctgcagttgcagcaa
gtgttttaattaacacaagtggatttttacagcaaaaagctgcaacaaca
ggaaaagaaagtactgaacaggttgcaagtggattacaagtaatgggcgt
aaatggataccaggatggaactaatgatgcaaatgtaagtaaaatggcaa
tttatgtaacccctaacgcaggaagttcagcaattgaccttacaaattca
aaattatttgtaacctacgatggccagacccacgtcttagcttacgatga
cgttacagaccttacaacaggtaattcagatattttcgatgcaattaatg
ttggaacccctgcttctgaattccacgttgcagtactccaggataatgat
aattcaactggaaatggagtaattaataaaggagatattgtagcaatagt
aattgaaactagcgacatttttggcaatgacggaattcctgaaagaaaga
gtgtttctggaaaagtacaaccggaatttggtgctccaggagtatttgaa
ttcacgacacctgcaacgtacactaacaaggtattggaattacaataa
(SEQ ID NO: 7)
Amino acid sequence
MSVKNFMNNKKGDSGIGTLIVFIAMVLVAAVAASVLINTSGFLQQKAATT
GKESTEQVASGLQVMGVNGYQDGTNDANVSKMAIYVTPNAGSSAIDLTNS
KLFVTYDGQTHVLAYDDVTDLTTGNSDIFDAINVGTPASEFHVAVLQDND
NSTGNGVINKGDIVAIVIETSDIFGNDGIPERKSVSGKVQPEFGAPGVFE
FTTPATYTNKVLELQ
(SEQ ID NO: 8)

Natrialba magadii Subunit1

Nucleotide sequence
Atgttcgaacaaaacgacgaccgcgaccgtggtcaggtggggattggcac
ccttatcgtgttcatcgcgatggtgcttgtcgctgcgattgccgcgggcg
tgctgatcaatacggctggcatgctgcagacgcaggcagaagccaccggt
gaagagagtacagatcaagtaagtgaccgcctggacatcgtcagtgtctc
aggggatgttgatgatcccgatgaccctactcaaatcaacaacatcagta
tggtgactgcgactgcgccgggatcggatccagttgacttgaatcaaaca
acggcgcagttcatcggtgagggtggtgaagagatgtttaatcttagcca
cgagggcgtcttcatcaacagcatccaaggcgtcacggatgaacccgata
acaacgtcttgacggaaagttcggaccgtgctgaagttgtgttcgaatta
gacggagccccaggtagttacgatattggctacgaagcattggatgagag
tgaacggttgacggttatcctgacgactgacgccggtgcgtccaccgaac
aggagattcgcgttccaagtaccttcattgaagacgaagaatcggtgaga
ctgtag
(SEQ ID NO: 9)
Amino acid sequence
MFEQNDDRDRGQVGIGTLIVFIAMVLVAAIAAGVLINTAGMLQTQAEATG
EESTDQVSDRLDIVSVSGDVDDPDDPTQINNISMVTATAPGSDPVDLNQT
TAQFIGEGGEEMFNLSHEGVFINSIQGVTDEPDNNVLTESSDRAEWFELD
GAPGSYDIGYEALDESERLTVILTTDAGASTEQEIRVPSTFIEDEESVRL
(SEQ ID NO: 10)

Natrialba magadii Subunit2

Nucleotide sequence
Atgttcactaacgacaccgacgacggccgcggtcaggtggggatcggcac
gctcatcgtgttcatcgcgatggtgctggtcgctgcgattgctgcgggcg
tcctgatgaacacagctgggatgttgcagtcccaggctgaagcaactggt
gaagagagtaccgaccttgtctctgaacggatcgataccacgatcgcagt
gggtaccgtatccacccatgtggcagacggtgaagacggtgcagatcgcg
gtgacttagcggagatcagtattggcgttaccggtgcacccggggcagat
gatattgacctcaatgagacgataattcaggtcgtcggtcctgagggggc
agagaatctcgtcatggctgacggaagcaatgacatgagtgaagctgggt
gggacgaaactagcaccaccgacattgggagtactgagagtactgaccaa
ggagatactgacgacgacgtaaacgcctcaaacatcgagagcggatactt
cgctgtcgaaaacgaagacggatactttgtcgagggtagcgatgcagtcc
tcgatgacaacaatggcgaactcacgatcgtcttcaatccaaaagtcgca
ccatttggtgaggctgatgatgtaagcggcatcacccctggagatcttca
tgaagatgacgtcttcggtgcgggcgacgaggcctcggtcgacatcgtct
cgccatccggtgcaaccacctcggtcgaactgaactccccagacctcttc
agcgagcctggtgaagcggtccgactctaa
(SEQ ID NO: 11)
Amino acid sequence
MFTNDTDDGRGQVGIGTLIVFIAMVLVAAIAAGVLMNTAGMLQSQAEATG
EESTDLVSERIDTTIAVGTVSTHVADGEDGADRGDLAEISIGVTGAPGAD
DIDLNETIIQVVGPEGAENLVMADGSNDMSEAGWDETSTTDIGSTESTDQ
GDTDDDVNASNIESGYFAVENEDGYFVEGSDAVLDDNNGELTIVFNPKVA
PFGEADDVSGITPGDLHEDDVFGAGDEASVDIVSPSGATTSVELNSPDLF
SEPGEAVRL
(SEQ ID NO: 12)

Natrialba magadii Subunit3

Nucleotide sequence
atgttcacatccaatacagatgacgaccgtggccaggtggggatcggtac
gctcatcgtgttcatcgcgatggtgctggtcgctgcgattgctgcgggcg
tattgatcaatacggctggcatgctgcagacgcaggccgaagccaccggc
gaagagagtacagatcaggtaagtgaccgacttgaaatctcgagtacgtc
tggagatttcagtgacgtaaatacccttggtgccggtgaaggcgaagaat
tggaggtaacggttgaagccggtgacgctacggcagcaggcgaagaagtc
gtaataagagttgcaacaagtgctgaatctggatttgaggactcgaaggc
aatcgaattacctgatgaagccggagatccaacaactgtcacacttgata
atcttccttcaataggtggtgcattggtaactgttgatggagaaaatgtt
caagcagtaacggaagacagtgtggacctcactcaaggagatccaagcgt
cagttttaacgtagacgaactcaggatgattccgagtcgactattggtct
tcaactcaccgctgatgctggtaacaacttctgggacgaaatagcggaag
ataatatcgaagataccgttactgttcagttgaccgactacgagcgtact
gaagctgaaataacgaacgtgaataattggggcagtgatgacgcagagat
tgagtgggaagcaacggtgccggctgacgagggagactatgcagtggaag
taataggattcgactcagcacggatgcttccaatttcgacaaatgaggta
gcaagtacaacggaagatccagaacttggtgaaactgacactcaaatcga
caaccttcagttctctgtcgctactgcacctggctctgacgcgatcgatc
ttgaggagacgtcagtgcagttcatcggtgatcagggcgaggagacggtt
acgatcactgaccggaacgtcgagaacatccagggtgtcgacggaaacgt
cctgacggataattccgatcgtgcactcgtctcgttcgacccagtcgccg
acattgacggattcaaccgaatcgaagagagcgaggacctcaccgtcata
ttcacgacggcatcgggagcctcgacagagaccgaactacgccattccaa
gcaccttcctcgaaggtgacgaatctgtgaggctataa
(SEQ ID NO: 13)
Amino acid sequence
MFTSNTDDDRGQVGIGTLIVFIAMVLVAAIAAGVLINTAGMLQTQAEATG
EESTDQVSDRLEISSTSGDFSDVNTLGAGEGEELEVTVEAGDATAAGEEV
VIRVATSAESGFEDSKAIELPDEAGDPTTVTLDNLPSIGGALVTVDGENV
QAVTEDSVDLTQGDPSVSFNVDELSDDSESTIGLQLTADAGNNFWDEIAE
DNIEDTVTVQLTDYERTEAEITNVNNWGSDDAEIEWEATVPADEGDYAVE
VIGFDSARMLPISTNEVASTTEDPELGETDTQIDNLQFSVATAPGSDAID
LEETSVQFIGDQGEETVTITDRNVENIQGVDGNVLTDNSDRALVSFDPVA
DIDGFNRIEESEDLTVIFTTASGASTETELRIPSTFLEGDESVRL
(SEQ ID NO: 14)

Natrialba magadii Subunit4

Nucleotide sequence
Atgtttgtcaacgaaactaccgacgaccgcggccaagtggggatcggtac
gctcatcgtgttcatcgcgatggtgctggtcgctgcgattgccgcaggtg
tactgatcaacacggccgggatgctgcaatcccaggccgaagcaaccggt
gaggagagtaccgatctcgtttccgaacggatcgattcaacgactgcagt
cggtattgtctccgaaaccgaagttagcgaggaggctggtgccgaccgag
gtgaactcgaagagattcgtcttggcgtcagcggtgctgctggctccgac
aatattgacctcagtgaaaccatcattcaggttgtgggccctcaaggaca
ggataaccttgtgatggctgatcctggtgatgatgaaatcgatgccaatg
atgatggattcgtcacggtgactgacgaagatggtaatgtggatggagac
agtactgatgcaactgatgctgacccatcccacattgcgtctggacactt
cgccgttgaaaacgaagatggcaatttcgtcgaggaaagcgatgcagtcc
tcgataacgacaacggcgaactcacgattatcctcaatccgaaggtagca
ccgttcggatcgcaaataagcgaaagtgatgaggaactagatctgcagga
tctggacactgaggacgcttcggtgctggagacgaatcctctctcagtat
cgtttcgccatccggtgcaacgacggaggtcgaactgaacgcgcctgacc
tcttcagcgaggacggcgaagcagttcgcctctaa
(SEQ ID NO: 15)
Amino acid sequence
MFVNETTDDRGQVGIGTLIVFIAMVLVAAIAAGVLINTAGMLQSQAEATG
EESTDLVSERIDSTTAVGIVSETEVSEEAGADRGELEEIRLGVSGAAGSD
NIDLSETIIQWGPQGQDNLVMADPGDDEIDANDDGFVTVTDEDGNVDGDS
TDATDADPSHIASGHFAVENEDGNFVEESDAVLDNDNGELTIILNPKVAP
FGSQISESDEELDLQDLDTEDAFGAGDESSLSIVSPSGATTEVELNAPDL
FSEDGEAVRL
(SEQ ID NO: 16)

Pyrococcus abysii FlaB1-1_b5

Nucleotide sequence
Atgaggagaggtgcgatcggcattggcacgttgatagttttcatcgcaat
ggttttagtagcggcagtagcagcgggagtgctcattagcacttctggat
atctccagcaaagggcaatgtctgtaggcctagagactacaagggatgtt
tcaagtggtctcagaataatctcaatctggggctatgcccctaagaatac
tactggcaataccaccattcagagcaatattaccaaactcgccatataca
tagctcccaacgctggaagtgaacccataaacctcaaccagacaaggata
atactcacagtaaagtcaacgatggtcatatttacctttggtggagagga
taccgttgcagactggacgaatggtgcagttaatgtctttaatgaaacca
tatgggaaaatattaacggaacaaagtttggagtgggagttgtggttgat
agcgataaaagcatgctttccaacaaggcatcaccgggaatgaactcggg
agatttagcagtactgctaattaacactaaattggcttttaacaaatacg
ggggaattccgcctaacacaaaggtggtcggtaagatactgccaccacac
ggtgcaggaactgttatcgacttaataactccagctacttactccagtga
gggtattgagctccagtg
(SEQ ID NO: 17)
Amino acid sequence
MRRGAIGIGTLIVFIAMVLVAAVAAGVLISTSGYLQQRAMSVGLETTRDV
SSGLRIISIWGYAPKNTTGNTTIQSNITKLAIYIAPNAGSEPINLNQTRI
ILTVKSTMVIFTFGGEDTVADWTNGAVNVFNETIWENINGTKFGVGVVVD
SDKSMLSNKASPGMNSGDLAVLLINTKLAFNKYGGIPPNTKVVGKILPPH
GAGTVIDLITPATYSSEGIELQ
(SEQ ID NO: 18)

Pyrococcus abysii FlaB1-2_b4

Nucleotide sequence
Atgcacagaaagggtgcaataggcataggaacgctcattgtcttcattgc
aatggttctagtagcggcagtagcggcgggagttatcattggaacagctg
gttatcttcaacagaaggcacaggctacaggcatgcagacaacccaagag
gtttccagtgggataaagatcatcaacatctatggttacgtaaactcctc
tgtccctagtaatggcacaataaccaagatggcaatattcgtctcaccta
acgcagggagtggggggatatccctcagtaacgtgaaaattgttctcagc
gatggcaagaaactcgttgtctataattatagcaagggattgctttatga
caaacagataagcgacttgttcaatgattctatcgttacgatatggaaca
acattaccgatacaaccttcggaatagcggtcattaacgacagtgggaac
aaaatggacaaagattatccaaacttagaatggggagataccgtggcact
actcctcaggacaacagtttttgaaacagaggataaccgtagaggaatcg
gtcctggtactaggatagttgggaaggtaattcccgaagttggggctgca
ggtgttatagacttcacaacaccctcaacatataactaccgggtgatggt
actccagtga
(SEQ ID NO: 19)
Amino acid sequence
MHRKGAIGIGTLIVFIAMVLVAAVAAGVIIGTAGYLQQKAQATGMQTTQE
VSSGIKIINIYGYVNSSVPSNGTITKMAIFVSPNAGSGGISLSNVKIVLS
DGKKLVVYNYSKGLLYDKQISDLFNDSIVTIWNNITDTTFGIAVINDSGN
KMDKDYPNLEWGDTVALLLRTTVFETEDNRRGIGPGTRIVGKVIPEVGAA
GVIDFTTPSTYNYRVMVLQ
(SEQ ID NO: 20)

Pyrococcus abysii FlaB1-3_b2

Nucleotide sequence
Ttgaaaaacctccaagggggtgcatggcaaatggcaagaagaggtgcgat
tggtattggtaccctaatagtgtttattgccatggtgttagtggctgcag
tagctgcagcagttctcataaacacgagcggcttcctccagactagggct
tcaacagtaggtaaggagcagaccaggcaagtttcgactggttttattct
caaggacgcctatgtaacaggcaccaatacgataaaccttctagtaaccc
taccaacggggagctatcccgtcgacattagcaggacagttataatcgta
aacggaaagcaactcacatatggtagtactgctaacaccacaaatttctc
tgcaaaacctctggtaggagagattaacggcgacattgtacaaccaggat
caacaattctcataacattcaatatgagtgagggttggaccgtcgctcgg
ggagaaatcgttcctaacgttggttcaccaactccattcactgtaaccaa
agatcttgatagtgttcccagtagctga
(SEQ ID NO: 21)
Amino acid sequence
MKNLQGGAWQMARRGAIGIGTLIVFIAMVLVAAVAAAVLINTSGFLQTRA
STVGKEQTRQVSTGFILKDAYVTGTNTINLLVTLPTGSYPVDISRTVIIV
NGKQLTYGSTANTTNFSAKPLVGEINGDIVQPGSTILITFNMSEGWTVAR
GEIVPNVGSPTPFTVTKDLDSVPSS
(SEQ ID NO: 22)

Pyrococcus horikoshii 1

Nucleotide sequence
Atgaggaggggtgctattggtattggaacgctcatcgtgttcatcgcaat
ggtattggtagctgcggtagctgctggagtgttaattacaaccagtggct
accttcagcagaaggccatggccactggtaggcagaccacccaggaagta
gcaagcggaatcagagtgagtggcatttatggctatactccttcaaaccc
tccaggaagtggaaagataacgaggctagtagtctacgttactccaaacg
ctggtagcggaggtattgatctcgcccatgttagagttgtattaagtgac
ggtaaaagaatggcagtgtataggtactatgattcagacaaagaccaagg
actccaagcaggctatttcctatatgcaggggatattgagaacatagtac
cctactttaacgatacagatgtactctcagtaagcaattatacaacggta
accagtgtcgctgatgtctggaagaatctatattatgcaatgacacaaga
caataagatgctctttggaattgtggtcgttgcagatgacgatgatagcc
taagcaatacagctcatcccacgcttgggtttggagacaaagccgcccta
atcttgtggacgataccattcgatgacgacaatgattacagcaatggcta
tggaataccattcacgactccatcgacttacacggataacctaatggagc
tccagtga
(SEQ ID NO: 23)
Amino acid sequence
MRRGAIGIGTLIVFIAMVLVAAVAAGVLITTSGYLQQKAMATGRQTTQEV
ASGIRVSGIYGYTPSNPPGSGKITRLVVYVTPNAGSGGIDLAHVRVVLSD
GKRMAVYRYYDSDKDQGLQAGYFLYAGDIENIVPYFNDTDVLSVSNYTTV
TSVADVWKNLYYAMTQDNKMLFGIVVVADDDDSLSNTAHPTLGFGDKAAL
ILWTIPFDDDNDYSNGYGIPPSTKVVGKVIPENGAGGVIDFTTPSTYTDN
LMELQ
(SEQ ID NO: 24)

Pyrococcus horikoshii 2

Nucleotide sequence
Gtgaagaaaggtgctgtgggtattggtacccttatagtgtttattgctat
ggtgttagtggctgcagtagctgctgcagtgctcatcaacacgagcggtt
acctccagcaaaagagccaggccactggtaggcagaccacccaggaagta
gcaagcggaatcaaagtaacaagagttgttggtaaagccgacagtgccac
caatccaacttatattcaagagttagctgtttacataacaccaaatgctg
gaagctccggaattgacttaactaaggtaaggataactctaagtgatgga
gccgagctaatgcagaaccttggagctacgataaagttcgataatggaag
tgttcaggtgtactttgatccaactgactggacatcagcagcaccaacag
taataattgatacaactaacaaggtcatagagatagtaaatgctactgta
gatagtaatgataatcatattaaacctgcgacagacagtaatgtcactat
aagctttgacactccagtgagcttatatgcctttgctaatccagtcagtg
acgtgttcgataatgatgcctttaacaacttaacgactaagactgacttt
ggaatagcagtgcttcaagacagcgatgggagcttagacaacaaggagta
tccaaccttaaccaaaggcgatctagtagtactcgctctgagggtaggag
ggactcagtcattaggatacagctctggagttagcaagatatcagtgata
tccacaacaactactgacgttttaacaaagcaatctagcgttaatgtcac
aattacatggacagcagtgtttggaaatggattcgacaccggaactaagg
ttactggaaaagtcattccagaatttggtgctcctggaatcatagagttc
acgactccatcaacttacacccagcaggtcattgagcttcagtga
(SEQ ID NO: 25)
Amino acid sequence
MKKGAVGIGTLIVFIAMVLVAAVAAAVLINTSGYLQQKSQATGRQTTQEV
ASGIKVTRVVGKADSATNPTYIQELAVYITPNAGSSGIDLTKVRITLSDG
QKQAIFKYRVGNSANELYFLAELMQNLGATIKFDNGSVQVYFDPTDWTSA
APTVIIDTTNKVIEIVNATVDSNDNHIKPATDSNVTISFDTPVSLYAFAN
PVSDVFDNDAFNNLTTKTDFGIAVLQDSDGSLDNKEYPTLTKGDLVVLAL
RVGGTQSLGYSSGVSKISVISTTTTDVLTKQSSVNVTITWTAVFGNGFDT
GTKVTGKVIPEFGAPGIIEFTTPSTYTQQVIELQ
(SEQ ID NO: 26)

Pyrococcus horikoshii 3

Nucleotide sequence
Atgaggaggggtgctgtgggtattggtacccttatagtgtttattgctat
ggtgttagtggctgcagtagctgctgcagtgctcatcaacacgagcggtt
acctccagcaaaagagccaggccactggtaggcagaccacccaggaagta
gcaagcggaatcaaagtaacaagtgttattggtcacgtagatacaacgaa
taatgccatagacaagctagcaatttatgtctcacccaatgctggaagtg
aaggtattgacctgagatatactaaaatagttctaaggagcaagagtcaa
gaggtttcactttactacaaccgcagtaattactacaatggggcagtaga
taacatatttgacatttcaggagtttggccttcaaatggctacaccttcg
gaatagttgtcattcaagatagtgacaactcagtccagcagaattatcca
acgcttaaccagggagatctggtagcactgactgtaaatgctaatgcagc
tctcggtggtataaagccaggaacttcaattagtggtgaggttattcctg
agcagggtgctcctggcgttatagaattcacaacaccaagcacatacacc
gaaactgttgtcgagttacaatga
(SEQ ID NO: 27)
Amino acid sequence
MRRGAVGIGTLIVFIAMVLVAAVAAAVLINTSGYLQQKSQATGRQTTQEV
ASGIKVTSVIGHVDTTNNAIDKLAIYVSPNAGSEGIDLRYTKIVLRSKSQ
EVSLYYNRSNYYNGAVDNIFDISGVWPSNGYTFGIVVIQDSDNSVQQNYP
TLNQGDLVALTVNANAALGGIKPGTSISGEVIPEQGAPGVIEFTTPSTYT
ETVVELQ
(SEQ ID NO: 28)

Pyrococcus horikoshii 4

Nucleotide sequence
Gtgacagtagtgccaaggaagggtgctgtgggtattggtacccttatagt
gtttattgctatggtgttagtggctgcagtagctgctgcagtgctcatca
acactagtggatacttgcaacagaaggcatcggggactggtagagagaca
actcaagaagtagcaagcggaatcaaggttgacagagtagtcggttatgc
tccggacataactggggacataacaagacttgctgtttacatctcaccga
atgccggaagctcagggattgacctaaacaaggttagggtaattctaagc
aatggacaaaaggaggtttcccttaagtacaactacgtctataatgctac
atccagcacccagacatacgttgcacttccacagggcaacatattcaatg
atattgttcttggagtaaatggaaccagtgaaaatgcagcttccacccag
gtaaacttcaactggtctctcctgacaggatcaacgttcggtttaatagt
gctccaagatgctgacggaagcgtgaaagcaagtactccaactctcaacc
agggagaccttgttatcatagctatcgatgtagacgcagcccttggagga
ataccaccaaggacttcaattactggtgaggttattcctgagcagggtgc
tcctggcgttatagaattcacaacaccaagcacatacacggcacatgtta
tggagcttcagtaa
(SEQ ID NO: 29)
Amino acid sequence
MTWPRKGAVGIGTLIVFIAMVLVAAVAAAVLINTSGYLQQKASGTGRETT
QEVASGIKVDRWGYAPDITGDITRLAVYISPNAGSSGIDLNKVRVILSNG
QKEVSLKYNYVYNATSSTQTYVALPQGNIFNDIVLGVNGTSENAASTQVN
FNWSLLTGSTFGLIVLQDADGSVKASTPTLNQGDLVIIAIDVDAALGGIP
PRTSITGEVIPEQGAPGVIEFTTPSTYTAHVMELQ
(SEQ ID NO: 30)

Pyrococcus horikoshii 5

Nucleotide sequence
Atgaggaagggagcaataggcattggtacactgatcgtctttatcgcaat
ggttctagtagccgcagtagccgcgggggtaatcataggaacagcaggtt
acctccagcagaaagcccaagcagcagggaggcaaacaacccaggaagtt
gcaagtggaataaagatcgtcaatgtattcggctacataaacgcaactcc
cccaagcaatggaacgatagtcaagatggccatcctggtaactcccaacg
ctgggagcagtggaattgacttaagcaacgttaagatagtgctcagcgat
gggaagaggttagcggtttacaactacagcggagtactatacacggggaa
gatactcgacctcttcaacttgacgatctggaagaataccagcaacggaa
ccttcagcattgcagtggttaatgacgttggttcaaagatggagaaccac
cacccaaccctcgagtggggtgacaccgttgcactgctcctcagaactga
cgatgtcttcgagtacgaaggtaagggtggaatagggccatccacaaaga
taatagggaaggtgattccggatgctggagctgctggagttatagacttc
acgactcccccaacgtttggctacaacgtgttagagttgcagtga
(SEQ ID NO: 31)
Amino acid sequence
MRKGAIGIGTLIVFIAMVLVAAVAAGVIIGTAGYLQQKAQMGRQTTQEVA
SGIKIVNVFGYINATPPSNGTIVKMAILVTPNAGSSGIDLSNVKIVLSDG
KRLAVYNYSGVLYTGKILDLFNLTIWKNTSNGTFSIAWNDVGSKMENHHP
TLEWGDTVALLLRTDDVFEYEGKGGIGPSTKIIGKVIPDAGAAGVIDFTT
PPTFGYNVLELQ
(SEQ ID NO: 32)

Sulfolobus solfataricus 1

Nucleotide sequence
Atgaactccaaaaagatgttaaaggaatacaacaaaaaagtgaaaaggaa
aggattagcgggattagacactgcaataatattaatagcatttataataa
ctgcatcagtattagcttacgtggctataaatatgggattatttgtgaca
cagaaagccaaatccactataaataaaggagaggagacagcgtcaacagc
actaacactatccggctctgtcctatatgctgttaactatccattaaata
ctagaagctactggatatactttacagtatctccaagttctggagtttct
agcgtggaattgtcgcccactactacagccatctcgtttactgcatctgc
agaaggagtgacgtactcaaatatatataaatacaccttattaacagtat
ccccatctgaactagcgaatgtcgtatacgcgaatggacagtacttagat
ctcgtaaatcagcagacaagtgcaggtcaaacatatgtatattatcctaa
tccttactatgcgttactagcacttaattacacactatataattattatc
ttagtacaaaaacaccatcaccaatatttattaatagtagcattctatct
ctatctagccttccatcatggttgaagaatgacaatagttttactttcac
tctcaatataagcggcaaactagttacttactatgtgtttgttaatcaga
catttgcatttacatatccagtggcaggagatccgttaatagggagtgct
atcgcccccgccggatcagtaataggagtaatacttttgtttggaccaga
tctaggaagtcatgtatttcaatatcagacaataacaatacaaattacac
caaatataggatctcctctcacaatatctgaatatatataccagccagag
ggtagcgtatcagtaatagggtga
(SEQ ID NO: 33)
Amino acid sequence
MNSKKMLKEYNKKVKRKGLAGLDTAIILIAFIITASVLAYVAINMGLFVT
QKAKSTINKGEETASTALTLSGSVLYAVNYPLNTRSYWIYFTVSPSSGVS
SVELSPTTTAISFTASAEGVTYSNIYKYTLLTVSPSELANVVYANGQYLD
LVNQQTSAGQTYVYYPNPYYALLALNYTLYNYYLSTKTPSPIFINSSILS
LSSLPSWLKNDNSFTFTLNISGKLVTYYVFVNQTFAFTYPVAGDPLIGSA
IAPAGSVIGVILLFGPDLGSHVFQYQTITIQITPNIGSPLTISEYIYQPE
GSVSVIG
(SEQ ID NO: 34)

Sulfolobus solfataricus 2

Nucleotide sequence
Atgaactccaaaaagatgttaaaggaatacaacaaaaaagtgaaaaggaa
aggattagcgggattagacactgcaataatattaatagcatttataataa
ctgcatcagtattagcttacgtggctataaatatgggattatttgtgaca
cagaaagccaaatccactataaataaaggagaggagacagcgtcaacagc
actaacactatccggctctgtcctatatgctgttaactatccattaaata
ctagaagctactggatatactttacagtatctccaagttctggagtttct
agcgtggaattgtcgcccactactacagccatctcgtttactgcatctgc
agaaggagtgacgtactcaaatatatataaatacaccttattaacagtat
ccccatctgaactagcgaatgtcgtatacgcgaatggacagtacttagat
ctcgtaaatcagcagacaagtgcaggtcaaacatatgtatattatcctaa
tccttactatgcgttactagcacttaattacacactatataattattatc
ttagtacaaaaacaccatcaccaatatttattaatagtagcattctatct
ctatctagccttccatcatggttgaagaatgacaatagttttactttcac
tctcaatataagcggcaaactagttacttactatgtgtttgttaatcaga
catttgcatttacatatccagtggcaggagatccgttaatagggagtgct
atcgcccccgccggatcagtaataggagtaatacttttgtttggaccaga
tctaggaagtcatgtatttcaatatcagacaataacaatacaaattacac
caaatataggatctcctctcacaatatctgaatatatataccagccagag
ggtagcgtatcagtaatagggtga
(SEQ ID NO: 35)
Amino acid sequence
MNSKKMLKEYNKKVKRKGLAGLDTAIILIAFIITASVLAYVAINMGLFVT
QKAKSTINKGEETASTALTLSGSVLYAVNYPLNTRSYWIYFTVSPSSGVS
SVELSPTTTAISFTASAEGVTYSNIYKYTLLTVSPSELANVVYANGQYLD
LVNQQTSAGQTYVYYPNPYYALLALNYTLYNYYLSTKTPSPIFINSSILS
LSSLPSWLKNDNSFTFTLNISGKLVTYYVFVNQTFAFTYPVAGDPLIGSA
IAPAGSVIGVILLFGPDLGSHVFQYQTITIQITPNIGSPLTISEYIYQPE
GSVSVIG
(SEQ ID NO: 36)

Thermococcus kodakaraensis B1

Nucleotide sequence
Atgaagaccagaacaaggaaaggtgcggttggtattggaaccctgattgt
tttcatagccatggttctagtggcggcagtggccgcggcagtgctgatca
acacgagcggctacctgcagcagaagagccaggctactggaagagagacc
acccaggaagtagccagcggaataaaggtcgagagagtcgtcggtaagac
agacctcccgtataccaacattggatccgattcaacggagcttgattaca
taaggcagctcgccatctacgtcagcccgaacgccggaagctcgggaatc
gacctcagcaacaccaaggtcattctcagcaacggtgagaaggaggccgt
tctcaagtacgctggtggaccggatgatgattacgacgcattcaccaagg
gcgtccagaacgacatttttgacctgtactttaagtattcatcagatggt
accaactggaataatgagcacagtggtctcgccgcttggaagaacctcta
ctacacgggtaccaaccacgacccggccaagaacttcggtatcatcgtca
tccaggacgccgacaacagcctcaccgaagactacccgaccctcaacaag
ggcgacctcgtagtcctcacggtcctcgttggaagccttgaggagtacac
aggtaatccttcaaatgacgacaatgctgtctacgaaactggtggcgcca
agtacgactacattgacgttaatggcaatagcgatactactgataccata
cagggcgtcttcggcgagggaatccccgccggtaccaagatcaccggtga
ggtcgttccggagttcggcgctcctggcgtcatcgagttcaccaccccga
gcacctacactgaggccgttatggagctccagtga
(SEQ ID NO: 37)
Amino acid sequence
MKTRTRKGAVGIGTLIVFIAMVLVAAVAAAVLINTSGYLQQKSQATGRET
TQEVASGIKVERVVGKTDLPYTNIGSDSTELDYIRQLAIYVSPNAGSSGI
DLSNTKVILSNGEKEAVLKYAGGPDDDYDAFTKGVQNDIFDLYFKYSSDG
TNWNNEHSGLAAWKNLYYTGTNHDPAKNFGIIVIQDADNSLTEDYPTLNK
GDLVVLTVLVGSLEEYTGNPSNDDNAVYETGGAKYDYIDVNGNSDTTDTI
QGVFGEGIPAGTKITGEWPEFGAPGVIEFTTPSTYTEAVMELQ
(SEQ ID NO: 38)

Thermococcus kodakaraensis B3

Nucleotide sequence
Atgaggttccttaagaagcgtggtgcggttggtattggaactttgatagt
gttcatcgccatggtgctcgttgcggcagttgccgcggcagtgctcatca
acaccagcggctacctccagcagaagagccagagcactggaaggcaaacc
accgaggaggtagccagcggaataaaggtaacgagcatcgttggctatgc
accatacgacgatagcaacaaggtgtacaagccaataagcaagcttgcca
tctacgtcagcccgaacgccggaagtgccggcatcgacatgaagaaggtc
agggtaatactcagcgacggcagtatcgaggccgtgttgaagtatgacaa
ttcggacgctgacagtgatggaacgcttgacaaagacgtcttcgccgtcg
gcatgcccgacaacgtgtttgaggatgacaccggcacaacggcctacgat
ggcgatcagtacatcacctggagcgaactcaacgacaagaccttcggcat
catagtcgtccaggacagcgacggctccctcaagccgctcaccccgaccc
tcaacaagggtgacatcgccataatcgccgtcagggttggcaattattac
gttgacagcaacggtaacctccaggcatactcacccacaccagatggcgt
cttcggcgaaggcatcaagcccaacacccacataaccggccaggtcgttc
cggagcacggtgcccctggcgtcattgacttcaccacaccgtcaacctat
acccagagcgtcatggagctccagtga
(SEQ ID NO: 39)
Amino acid sequence
MRFLKKRGAVGIGTLIVFIAMVLVAAVAAAVLINTSGYLQQKSQSTGRQT
TEEVASGIKVTSIVGYAPYDDSNKVYKPISKLAIYVSPNAGSAGIDMKKV
RVILSDGSIEAVLKYDNSDADSDGTLDKDVFAVGMPDNVFEDDTGTTAYD
GDQYITWSELNDKTFGIIWQDSDGSLKPLTPTLNKGDIAIIAVRVGNYYV
DSNGNLQAYSPTPDGVFGEGIKPNTHITGQVVPEHGAPGVIDFTTPSTYT
QSVMELQ
(SEQ ID NO: 40)

Thermococcus kodakaraensis B4

Nucleotide sequence
Atgcgtaggaggggagcaataggcatcggcacgctgatcgtcttcatcgc
aatggtgctggttgctgcagtggccgcaggggtcatcatcggtacagcgg
gctaccttgagcagaaggctcaggccgctggcaggcagaccacacaggag
gtagccagcggaataaaggtgctcaacgtctacggctacaccaacgccac
acccccgagcaacggcacaatagagaggatggctatcttcataactccca
acgcaggcagtgagggcatcgacctgagcaacgttaagataguctcagcg
acggaaggaggctggtcgtttacaactactcgggtagcttccagaacgcc
gagagcgttaaggacctcttcaacatgacctacgttggcgtgtggaacag
cacaaatggaacggccagctttggcatagccgtcatcaacgacataggca
gcgagatgcagggaacccacccgacgcttgagttcggtgacatggtcgcg
ctatgcgtctggacgacgatgttcgagtacgaggataaggacggcatagg
cccgagcaccaggataaccggaaaggtcatccccgagaggggcgccgccg
gtgtgctcgacttcaccacgccggccacgttcagctacaacgtgatggtg
ctccagtga
(SEQ ID NO: 41)
Amino acid sequence
MRRRGAIGIGTLIVFIAMVLVAAVAAGVIIGTAGYLEQKAQAAGRQTTQE
VASGIKVLNVYGYTNATPPSNGTIERMAIFITPNAGSEGIDLSNVKIVLS
DGRRLVVYNYSGSFQNAESVKDLFNMTYVGVWNSTNGTASFGIAVINDIG
SEMQGTHPTLEFGDMVALCVWTTMFEYEDKDGIGPSTRITGKVIPERGAA
GVLDFTTPATFSYNVMVLQ
(SEQ ID NO: 42)

Thermococcus kodakaraensis B5

Nucleotide sequence
Atgaggaggggagcaataggcattggaacgctcatcgtgttcattgccat
ggtgctggttgccgcggtggccgctggagtgctcataagcaccagcggct
acctccagcagaaggcaatgagcgccggcaggcagaccacccaggaggtc
gcgagcggaattaaggtgctcaacgtctacggctacatcaacggttcaac
acccggcgcccacaatataaccagactcgtcctctacgtcagcccgaatg
ccggatccggtggcattgaccttgcccacgttaaggtcgtcataagcgac
ggcaagaggatggccgtttatcgttactacgaccccaatgaggacaaaaa
cagtgatatccagccagcttacatccactacacaggggacatcgctaacg
tctttgcctatgagaagtgggagccgtactataaaggtaagtaccccacc
gggtttgaccccaacaataagttctacataacggacaacatcgacataag
cgccgtctggtggaacctctacagcgcctacaacaagaccagtaataatg
ataaggactacggtaaactcctctttggaattgcggtcgttcaggacggt
gacgagagccttgacagtgagaaccaccccagcctcagctggggtgacat
agcggccattatgctgtggacgttcccgtttgatgataacaacaatccga
tcgatggattcggtctgccaccgagcaccaaggtcaccggaaaggtcata
cctgagaacggtgcgggcggcgtcatagacttcacaacaccatcgacgta
tactgacaacatactggaactccagtga
(SEQ ID NO: 43)
Amino acid sequence
MRRGAIGIGTLIVFIAMVLVAAVAAGVLISTSGYLQQKAMSAGRQTTQEV
ASGIKVLNVYGYINGSTPGAHNITRLVLYVSPNAGSGGIDLAHVKWISDG
KRMAVYRYYDPNEDKNSDIQPAYIHYTGDIANVFAYEKWEPYYKGKYPTG
FDPNNKFYITDNIDISAVWWNLYSAYNKTSNNDKDYGKLLFGIAWVVDGD
ESLDSENHPSLSWGDIAAIMLWTFPFDDNNNPIDGFGLPPSTKVTGKVIP
ENGAGGVIDFTTPSTYTDNILELQ
(SEQ ID NO: 44)

1. An adhesive material being composed and/or consisting of at least one protein obtained or obtainable from flagella from archaea.

2. Use of at least one protein obtained or obtainable from flagella from archaea for the preparation of an adhesive material.

3. A method for the preparation of an adhesive material comprising the step of isolating and/or purifying at least one protein obtained from flagella from archaea.

4. The adhesive material of claim 1, the use of claim 2 or the method of claim 3, whereby said at least one protein obtained from flagella from archaea is recombinantly produced, chemically isolated from flagella or chemically synthesized.

5. The adhesive material of claim 1, the use of claim 2 or the method of claim 3, whereby said protein is a flagellin.

6. The adhesive material, the use or the method of claim 5, whereby said flagellin is a flagellin obtained and/or derived from A. fulgidus, A. pernix, H. salinarum, M. jannaschii, M. maripaludis, M. vannielii, M. voltae, P. abysii P. horikoshii, P. kodakarensis, P. furiosus.

7. The adhesive material, the use or the method of claim of any one of claims 4 to 6, whereby said flagellin is encoded by a polynucleotide selected from the group consisting of
(a) a polynucleotide having a nucleotide sequence encoding the polypeptide having the deduced amino acid sequence as shown in SEQ ID NOs:2, 4, 6, 8, 10, 12, 14, 16, 18, 20, 22, 24, 26, 28, 30, 32, 34, 36, 38, 40, 42 or 44;
(b) a polynucleotide having the coding sequence as shown in SEQ ID NOs:1, 3, 5, 7, 9, 11, 13, 15, 17, 19, 21, 23, 25, 27, 29, 31, 33, 35, 37, 39, 41 or 43;
(c) a polynucleotide having a nucleotide sequence encoding a fragment or derivative of a polypeptide encoded by a polynucleotide of any one of (a) or (b), wherein in said derivative one or more amino acid residues are conservatively substituted compared to said polypeptide, and said fragment or derivative encodes an archaeal flagellin;
(d) a polynucleotide having a nucleotide sequence which is at least 70% identical to a polynucleotide as defined in any one of (a) to (c) and which encodes an archaeal flagellin;
(e) a polynucleotide having a nucleotide sequence the complementary strand of which hybridizes to a polynucleotide as defined in any one of (a) to (d) and which encodes an archaeal flagellin; and
(e) a polynucleotide having a nucleotide sequence being degenerate to the nucleotide sequence of the polynucleotide of any one of (a) to (e);
or the complementary strand of such a polynucleotide.

8. A method for the production of a polypeptide encoded by the polynucleotide as defined in claim 7 comprising culturing a host cell comprising said polynucleotide and recovering said polypeptide.

9. A polypeptide encoded by the polynucleotide as defined in claim 7 or obtainable by the method of claim 8.

10. The adhesive material, the use or the method of claim of any one of claim 4 to 7, whereby said flagellin comprises in its amino acid sequence the consensus sequence AxGIGTLIVFIAMVLVAAVAA.

11. The adhesive material, the use or the method of any one of claims 4 to 7 or 10, whereby said flagellin is obtainable by
(a) culturing archaea cells with flagella;
(b) shearing the flagella from said cells;
(c) purifying said flagella;
(d) isolating the flagellin from said flagella by using denaturing agents

12. The adhesive material, the use or the method of any one of claims 4 to 7, 10 or 11, whereby said flagellin is obtainable from Pyrococcus furiosus (P. furiosus).

13. The adhesive material, the use or the method of claim 12, whereby said P. furiosus is P. furiosus Vc1.

14. The adhesive material, the use or the method of claim 13, whereby said P. furiosus Vc1 is deposited under DSM3638.

15. The adhesive material, the use or the method of any one of claims 12 to 14, whereby said flagellin is a 30 kDa protein.

16. The adhesive material, the use or the method of any one of claims 12 to 15, whereby said flagellin is encoded by a nucleotide sequence as shown in SEQ ID NO: 1 or wherein said flagellin is or comprises an amino acid sequence as shown in SEQ ID NO: 2.

17. A composition comprising the adhesive material of any one of claims 1, 3 to 7 or 10 to 16, at least one protein as defined in claim 2 or the protein of claim 9.

18. The composition of claim 17 which is a pharmaceutical composition.
